(19) United States
(12) Patent Application Publication
(10) Pub. No.: US 2026/0340919 A1
(43) Pub. Date: Oct. 29, 2026

(54) SYSTEMS AND METHODS FOR PRESENTING DYNAMIC AVATARS

(71) Applicant: RESMED DIGITAL HEALTH INC., San Diego, CA (US)

(72) Inventors: Michael James DENT, Bella Vista, NSW (AU); James Douglas WEBSTER, Bella Vista, NSW (AU); Amila Jeewaka FERNANDO, Bella Vista, NSW (AU); Wendy Wen Yi LEONG, Singapore (SG); Albert Jack Greenwood WOFFENDEN, Bella Vista, NSW (AU); Siew Ting OOI, Singapore (SG)

(21) Appl. No.: 19/104,946

(22) PCT Filed: Aug. 16, 2023
(86) PCT No.: PCT/US2023/030384

§ 371 Date: Feb. 19, 2025

Related U.S. Application Data
(60) Provisional application No. 63/399,403, filed on Aug. 19, 2022.

(51) Int. Cl. G06T 13/40; A61B 5/00; G16H 50/30

(52) U.S. Cl. CPC G06T 13/40; A61B 5/4818; A61B 5/4833; A61B 5/486; A61B 5/744; G06T 2210/41; G16H 50/30

(57) ABSTRACT

A method includes receiving sensor data from one or more sensors. The sensor data is associated with a sleep session of an individual. The method further includes determining sleep data for the sleep session based at least in part on the sensor data. The method further includes accessing avatar information associated a digital avatar that is associated with the individual. The method further includes updating the avatar information based at least in part on the sleep data. The method further includes generating and presenting a display based at least in part on the updated avatar information.

### CROSS-REFERENCE TO RELATED APPLICATIONS

This application claims the benefit of, and priority to, U.S. Provisional Patent Application No. 63/399,403, filed Aug. 19, 2022, which is hereby incorporated by reference herein in its entirety.

### TECHNICAL FIELD

The present disclosure relates generally to systems and methods for updating and presenting digital avatars and related information, and more particularly, to systems and methods for dynamically presenting digital avatars and related information based on sleep data.

### BACKGROUND

Many individuals suffer from sleep-related and/or respiratory-related disorders such as, for example, Sleep Disordered Breathing (SDB), which can include Obstructive Sleep Apnea (OSA), Central Sleep Apnea (CSA), other types of apneas such as mixed apneas and hypopneas, Respiratory Effort Related Arousal (RERA), and snoring. In some cases, these disorders manifest, or manifest more pronouncedly, when the individual is in a particular lying/sleeping position. These individuals may also suffer from other health conditions (which may be referred to as comorbidities), such as insomnia (e.g., difficulty initiating sleep, frequent or prolonged awakenings after initially falling asleep, and/or an early awakening with an inability to return to sleep), Periodic Limb Movement Disorder (PLMD), Restless Leg Syndrome (RLS), Cheyne-Stokes Respiration (CSR), respiratory insufficiency, Obesity Hyperventilation Syndrome (OHS), Chronic Obstructive Pulmonary Disease (COPD), Neuromuscular Disease (NMD), rapid eye movement (REM) behavior disorder (also referred to as RBD), dream enactment behavior (DEB), hypertension, diabetes, stroke, and chest wall disorders.

These disorders are often treated using a respiratory therapy system (e.g., a continuous positive airway pressure (CPAP) system), which delivers pressurized air to aid in preventing the individual's airway from narrowing or collapsing during sleep. However, some users find such systems to be uncomfortable, difficult to use, expensive, aesthetically unappealing and/or fail to perceive the benefits associated with using the system. As a result, some users will elect not to use the respiratory therapy system or discontinue use of the respiratory therapy system absent a demonstration of the severity of their symptoms when respiratory therapy treatment is not used or encouragement or affirmation that the respiratory therapy system is improving their sleep quality and reducing the symptoms of these disorders. The present disclosure is directed to solving these and other problems.

### SUMMARY

According to some implementations of the present disclosure, a method includes receiving sensor data from one or more sensors. The sensor data is associated with a sleep session of an individual. The method further includes determining sleep data for the sleep session based at least in part on the sensor data. The method further includes accessing avatar information associated a digital avatar that is associated with the individual. The method further includes updating the avatar information based at least in part on the sleep data. The method further includes generating and presenting a display based at least in part on the updated avatar information.

According to some implementations of the present disclosure, a system includes an electronic interface, a memory, and a control system. The control system is configured to receive sensor data from one or more sensors. The sensor data is associated with a sleep session of an individual. The memory stores machine-readable instructions. The control system includes one or more processors configured to execute the machine-readable instructions to determine sleep data for the sleep session from the sensor data. The control system is further configured to access avatar information associated a digital avatar that is associated with the individual. The control system is further configured to update the avatar information based at least in part on the sleep data. The control system is further configured to generate and present a display based at least in part on the updated avatar information.

The above summary is not intended to represent each implementation or every aspect of the present disclosure. Additional features and benefits of the present disclosure are apparent from the detailed description and figures set forth below.

### BRIEF DESCRIPTION OF THE DRAWINGS

FIG. 1 is a functional block diagram of a system, according to some implementations of the present disclosure.

FIG. 2 is a perspective view of at least a portion of the system of FIG. 1, a user, and a bed partner, according to some implementations of the present disclosure.

FIG. 3 illustrates an exemplary timeline for a sleep session, according to some implementations of the present disclosure.

FIG. 4 illustrates an exemplary hypnogram associated with the sleep session of FIG. 3, according to some implementations of the present disclosure.

FIG. 5 is a flowchart depicting a process for updating avatar information based on sleep data according to certain aspects of the present disclosure.

FIG. 6 is a flowchart depicting a process for presenting displays related to milestone achievements based on sleep data according to certain aspects of the present disclosure.

FIG. 7 is a flowchart depicting a process for managing earned achievements based on sleep data according to certain aspects of the present disclosure.

FIG. 8 is a flowchart depicting a process for presenting a digital avatar with a sleep accessory according to certain aspects of the present disclosure.

FIG. 9 is a flowchart depicting a process for using facial scan data according to certain aspects of the present disclosure.

FIG. 10 is an illustration of a graphical user interface displaying a digital avatar following a high-quality sleep session, according to certain aspects of the present disclosure.

FIG. 11 is an illustration of a graphical user interface displaying a digital avatar following a low-quality sleep session, according to certain aspects of the present disclosure.

While the present disclosure is susceptible to various modifications and alternative forms, specific implementations and embodiments thereof have been shown by way of example in the drawings and will herein be described in detail. It should be understood, however, that it is not intended to limit the present disclosure to the particular forms disclosed, but on the contrary, the present disclosure is to cover all modifications, equivalents, and alternatives falling within the spirit and scope of the present disclosure as defined by the appended claims.

### DETAILED DESCRIPTION

Certain aspects and features of the present disclosure relate to leveraging sleep data (e.g., hours slept, quality of sleep, time spent in certain sleep stages, respiratory therapy usage, etc.) to dynamically update a digital avatar and information related to the digital avatar. An individual's sleep session can be tracked, permitting the digital avatar to be customized, when next presented, according to that sleep session. The digital avatar can help encourage the individual to meet certain goals (e.g., sleeping better, having more energy, using respiratory therapy more often, etc.). The digital avatar can be used to interact with others in a virtual environment. Achievements can be earned and tracked in association with the digital avatar, and in some cases, be redeemed for virtual or real-world incentives.

Many individuals suffer from sleep-related and/or respiratory disorders, such as Sleep Disordered Breathing (SDB) such as Obstructive Sleep Apnea (OSA), Central Sleep Apnea (CSA) and other types of apneas, Respiratory Effort Related Arousal (RERA), snoring, Cheyne-Stokes Respiration (CSR), respiratory insufficiency, Obesity Hyperventilation Syndrome (OHS), Chronic Obstructive Pulmonary Disease (COPD), Periodic Limb Movement Disorder (PLMD), Restless Leg Syndrome (RLS), Neuromuscular Disease (NMD), and chest wall disorders.

Obstructive Sleep Apnea (OSA), a form of Sleep Disordered Breathing (SDB), is characterized by events including occlusion or obstruction of the upper air passage during sleep resulting from a combination of an abnormally small upper airway and the normal loss of muscle tone in the region of the tongue, soft palate and posterior oropharyngeal wall. More generally, an apnea generally refers to the cessation of breathing caused by blockage of the air (Obstructive Sleep Apnea) or the stopping of the breathing function (often referred to as Central Sleep Apnea). CSA results when the brain temporarily stops sending signals to the muscles that control breathing. Typically, the individual will stop breathing for between about 15 seconds and about 30 seconds during an obstructive sleep apnea event.

Other types of apneas include hypopnea, hyperpnea, and hypercapnia. Hypopnea is generally characterized by slow or shallow breathing caused by a narrowed airway, as opposed to a blocked airway. Hyperpnea is generally characterized by an increase depth and/or rate of breathing. Hypercapnia is generally characterized by elevated or excessive carbon dioxide in the bloodstream, typically caused by inadequate respiration.

A Respiratory Effort Related Arousal (RERA) event is typically characterized by an increased respiratory effort for ten seconds or longer leading to arousal from sleep and which does not fulfill the criteria for an apnea or hypopnea event. RERAs are defined as a sequence of breaths characterized by increasing respiratory effort leading to an arousal from sleep, but which does not meet criteria for an apnea or hypopnea. These events fulfil the following criteria: (1) a pattern of progressively more negative esophageal pressure, terminated by a sudden change in pressure to a less negative level and an arousal, and (2) the event lasts ten seconds or longer. In some implementations, a Nasal Cannula/Pressure Transducer System is adequate and reliable in the detection of RERAs. A RERA detector may be based on a real flow signal derived from a respiratory therapy device. For example, a flow limitation measure may be determined based on a flow signal. A measure of arousal may then be derived as a function of the flow limitation measure and a measure of sudden increase in ventilation. One such method is described in WO 2008/138040 and U.S. Pat. No. 9,358,353, assigned to ResMed Ltd., the disclosure of each of which is hereby incorporated by reference herein in their entireties.

Cheyne-Stokes Respiration (CSR) is another form of sleep disordered breathing. CSR is a disorder of a patient's respiratory controller in which there are rhythmic alternating periods of waxing and waning ventilation known as CSR cycles. CSR is characterized by repetitive de-oxygenation and re-oxygenation of the arterial blood.

Obesity Hyperventilation Syndrome (OHS) is defined as the combination of severe obesity and awake chronic hypercapnia, in the absence of other known causes for hypoventilation. Symptoms include dyspnea, morning headache and excessive daytime sleepiness.

Chronic Obstructive Pulmonary Disease (COPD) encompasses any of a group of lower airway diseases that have certain characteristics in common, such as increased resistance to air movement, extended expiratory phase of respiration, and loss of the normal elasticity of the lung. COPD encompasses a group of lower airway diseases that have certain characteristics in common, such as increased resistance to air movement, extended expiratory phase of respiration, and loss of the normal elasticity of the lung.

Neuromuscular Disease (NMD) encompasses many diseases and ailments that impair the functioning of the muscles either directly via intrinsic muscle pathology, or indirectly via nerve pathology. Chest wall disorders are a group of thoracic deformities that result in inefficient coupling between the respiratory muscles and the thoracic cage.

These and other disorders are characterized by particular events (e.g., snoring, an apnea, a hypopnea, a restless leg, a sleeping disorder, choking, an increased heart rate, labored breathing, an asthma attack, an epileptic episode, a seizure, or any combination thereof) that occur when the individual is sleeping.

The Apnea-Hypopnea Index (AHI) is an index used to indicate the severity of sleep apnea during a sleep session. The AHI is calculated by dividing the number of apnea and/or hypopnea events experienced by the user during the sleep session by the total number of hours of sleep in the sleep session. The event can be, for example, a pause in breathing that lasts for at least 10 seconds. An AHI that is less than 5 is considered normal. An AHI that is greater than or equal to 5, but less than 15 is considered indicative of mild sleep apnea. An AHI that is greater than or equal to 15, but less than 30 is considered indicative of moderate sleep apnea. An AHI that is greater than or equal to 30 is considered indicative of severe sleep apnea. In children, an AHI that is greater than 1 is considered abnormal. Sleep apnea can be considered “controlled” when the AHI is normal, or when the AHI is normal or mild. The AHI can also be used in combination with oxygen desaturation levels to indicate the severity of Obstructive Sleep Apnea.

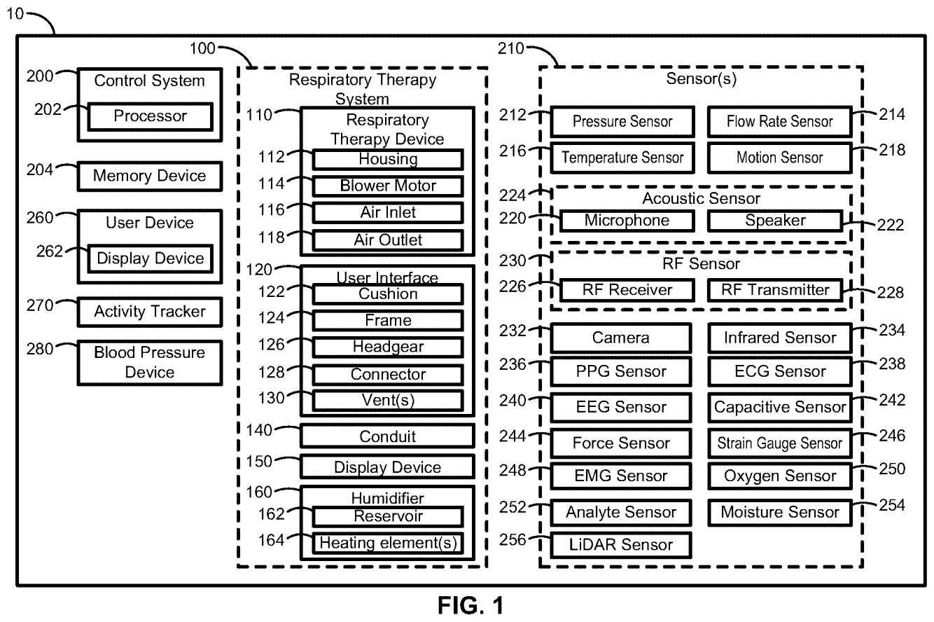

Referring to FIG. 1, a system 10, according to some implementations of the present disclosure, is illustrated. The system 10 includes a respiratory therapy system 100, a control system 200, one or more sensors 210, a user device 260, and an activity tracker 270.

The respiratory therapy system 100 includes a respiratory pressure therapy (RPT) device 110 (referred to herein as respiratory therapy device 110), a user interface 120 (also referred to as a mask or a patient interface), a conduit 140 (also referred to as a tube or an air circuit), a display device 150, and a humidifier 160. Respiratory pressure therapy refers to the application of a supply of air to an entrance to a user's airways at a controlled target pressure that is nominally positive with respect to atmosphere throughout the user's breathing cycle (e.g., in contrast to negative pressure therapies such as the tank ventilator or cuirass). The respiratory therapy system 100 is generally used to treat individuals suffering from one or more sleep-related respiratory disorders (e.g., obstructive sleep apnea, central sleep apnea, or mixed sleep apnea).

The respiratory therapy system 100 can be used, for example, as a ventilator or as a positive airway pressure (PAP) system, such as a continuous positive airway pressure (CPAP) system, an automatic positive airway pressure system (APAP), a bi-level or variable positive airway pressure system (BPAP or VPAP), or any combination thereof. The CPAP system delivers a predetermined air pressure (e.g., determined by a sleep physician) to the user. The APAP system automatically varies the air pressure delivered to the user based on, for example, respiration data associated with the user. The BPAP or VPAP system is configured to deliver a first predetermined pressure (e.g., an inspiratory positive airway pressure or IPAP) and a second predetermined pressure (e.g., an expiratory positive airway pressure or EPAP) that is lower than the first predetermined pressure.

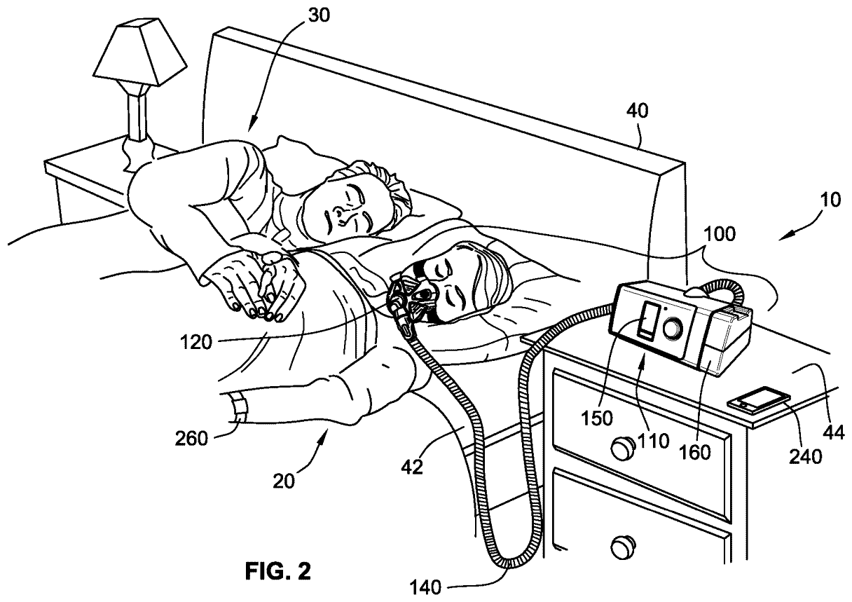

As shown in FIG. 2, the respiratory therapy system 100 can be used to treat user 20. In this example, the user 20 of the respiratory therapy system 100 and a bed partner 30 are located in a bed 40 and are laying on a mattress 42. The user interface 120 can be worn by the user 20 during a sleep session. The respiratory therapy system 100 generally aids in increasing the air pressure in the throat of the user 20 to aid in preventing the airway from closing and/or narrowing during sleep. The respiratory therapy device 110 can be positioned on a nightstand 44 that is directly adjacent to the bed 40 as shown in FIG. 2, or more generally, on any surface or structure that is generally adjacent to the bed 40 and/or the user 20.

The respiratory therapy device 110 is generally used to generate pressurized air that is delivered to a user (e.g., using one or more motors that drive one or more compressors). In some implementations, the respiratory therapy device 110 generates continuous constant air pressure that is delivered to the user. In other implementations, the respiratory therapy device 110 generates two or more predetermined pressures (e.g., a first predetermined air pressure and a second predetermined air pressure). In still other implementations, the respiratory therapy device 110 generates a variety of different air pressures within a predetermined range. For example, the respiratory therapy device 110 can deliver at least about 6 cmH₂O, at least about 10 cmH₂O, at least about 20 cmH₂O, between about 6 cmH₂O and about 10 cmH₂O, between about 7 cmH₂O and about 12 cmH₂O, etc. The respiratory therapy device 110 can also deliver pressurized air at a predetermined flow rate between, for example, about −20 L/min and about 150 L/min, while maintaining a positive pressure (relative to the ambient pressure).

Referring back to FIG. 1, the respiratory therapy device 110 includes a housing 112, a blower motor 114, an air inlet 116, and an air outlet 118. The blower motor 114 is at least partially disposed or integrated within the housing 112. The blower motor 114 draws air from outside the housing 112 (e.g., atmosphere) via the air inlet 116 and causes pressurized air to flow through the humidifier 160, and through the air outlet 118. In some implementations, the air inlet 116 and/or the air outlet 118 include a cover that is moveable between a closed position and an open position (e.g., to prevent or inhibit air from flowing through the air inlet 116 or the air outlet 118). In some cases, the housing 112 can include a vent 113 to allow air to pass through the housing 112 to the air inlet 116. As described below, the conduit 140 is coupled to the air outlet 118 of the respiratory therapy device 110.

The user interface 120 engages a portion of the user's face and delivers pressurized air from the respiratory therapy device 110 to the user's airway to aid in preventing the airway from narrowing and/or collapsing during sleep. This may also increase the user's oxygen intake during sleep. Generally, the user interface 120 engages the user's face such that the pressurized air is delivered to the user's airway via the user's mouth, the user's nose, or both the user's mouth and nose. Together, the respiratory therapy device 110, the user interface 120, and the conduit 140 form an air pathway fluidly coupled with an airway of the user. The pressurized air also increases the user's oxygen intake during sleep. Depending upon the therapy to be applied, the user interface 120 may form a seal, for example, with a region or portion of the user's face, to facilitate the delivery of gas at a pressure at sufficient variance with ambient pressure to effect therapy, for example, at a positive pressure of about 10 cm H₂O relative to ambient pressure. For other forms of therapy, such as the delivery of oxygen, the user interface may not include a seal sufficient to facilitate delivery to the airways of a supply of gas at a positive pressure of about 10 cmH₂O.

The user interface 120 can include, for example, a cushion 122, a frame 124, a headgear 126, connector 128, and one or more vents 130. The cushion 122 and the frame 124 define a volume of space around the mouth and/or nose of the user. When the respiratory therapy system 100 is in use, this volume space receives pressurized air (e.g., from the respiratory therapy device 110 via the conduit 140) for passage into the airway(s) of the user. The headgear 126 is generally used to aid in positioning and/or stabilizing the user interface 120 on a portion of the user (e.g., the face), and along with the cushion 122 (which, for example, can comprise silicone, plastic, foam, etc.) aids in providing a substantially air-tight seal between the user interface 120 and the user 20. In some implementations the headgear 126 includes one or more straps (e.g., including hook and loop fasteners). The connector 128 is generally used to couple (e.g., connect and fluidly couple) the conduit 140 to the cushion 122 and/or frame 124. Alternatively, the conduit 140 can be directly coupled to the cushion 122 and/or frame 124 without the connector 128. The vent 130 can be used for permitting the escape of carbon dioxide and other gases exhaled by the user 20. The user interface 120 generally can include any suitable number of vents (e.g., one, two, five, ten, etc.).

In some implementations, the user interface 120 is a facial mask (e.g., a full face mask) that covers at least a portion of the nose and mouth of the user 20. Alternatively, the user interface 120 can be a nasal mask that provides air to the nose of the user or a nasal pillow mask that delivers air directly to the nostrils of the user 20. In other implementations, the user interface 120 includes a mouthpiece (e.g., a night guard mouthpiece molded to conform to the teeth of the user, a mandibular repositioning device, etc.).

In some cases, the cushion 122 and frame 124 of the user interface 120 form a unitary component of the user interface 120. The user interface 120 can also include a headgear 126, which generally includes a strap assembly and optionally a connector 128. The headgear 126 can be configured to be positioned generally about at least a portion of a user's head when the user wears the user interface 120. The headgear 126 can be coupled to the frame 124 and positioned on the user's head such that the user's head is positioned between the headgear 126 and the frame 124. The cushion 122 can be positioned between the user's face and the frame 124 to form a seal on the user's face. The optional connector 128 can be configured to couple to the frame 124 and/or cushion 122 at one end and to a conduit 140 of a respiratory therapy system 100. The pressurized air can flow directly from the conduit 140 of the respiratory therapy system 100 into the volume of space defined by the cushion 122 (or cushion 122 and frame 124) of the user interface 120 through the connector 128. From the user interface 120, the pressurized air reaches the user's airway through the user's mouth, nose, or both. Alternatively, where the user interface 120 does not include the connector 128, the conduit of the respiratory therapy system can connect directly to the cushion 122 and/or the frame 124.

In some implementations, the connector 128 may include one or more vents 130 (e.g., a plurality of vents) located on the main body of the connector 128 itself and/or one or a plurality of vents 130 (“diffuser vents”) in proximity to the frame 124, for permitting the escape of carbon dioxide (CO₂) and other gases exhaled by the user. In some implementations, one or a plurality of vents 130 may be located in the user interface 120, such as in frame 124, and/or in the conduit 140. In some implementations, the frame 124 includes at least one anti-asphyxia valve (AAV), which allows CO₂ and other gases exhaled by the user to escape in the event that the vents 130 fail when the respiratory therapy device is active. In general, AAVs are present for full face masks (e.g., as a safety feature); however, the diffuser vents and vents located on the mask or connector (usually an array of orifices in the mask material itself or a mesh made of some sort of fabric, in many cases replaceable) are not necessarily both present (e.g., some masks might have only the diffuser vents such as the plurality of vents 130, other masks might have only the plurality of vents 130 on the connector 128 itself).

In some cases, the user interface 120 can be an indirect user interface. Such an interface 120 can include a headgear 126 (e.g., as a strap assembly), a cushion 122, a frame 124, a connector 128, and a user interface conduit (often referred to as a minitube or a flexitube). The user interface 120 is an indirectly connected user interface because pressurized air is delivered from the conduit 140 of the respiratory therapy system to the cushion 122 and/or frame 124 through the user interface conduit, rather than directly from the conduit 140 of the respiratory therapy system.

In some implementations, the cushion 122 and frame 124 form a unitary component of the user interface 120. Generally, the user interface conduit is more flexible than the conduit 140 of the respiratory therapy system 100 described above and/or has a diameter smaller than the diameter of the than the than the conduit 140. The user interface conduit is typically shorter that conduit 140. The headgear 126 of such a user interface 120 can be configured to be positioned generally about at least a portion of a user's head when the user wears the user interface 120. The headgear 126 can be coupled to the frame 124 and positioned on the user's head such that the user's head is positioned between the headgear 126 and the frame 124. The cushion 122 is positioned between the user's face and the frame 124 to form a seal on the user's face. The connector 128 is configured to couple to the frame 124 and/or cushion 122 at one end and to the conduit of the user interface 120 at the other end. In other implementations, the user interface conduit may connect directly to frame 124 and/or cushion 122. The user interface conduit, at the opposite end relative to the frame 124 and cushion 122, is configured to connect to the conduit 140. The pressurized air can flow from the conduit 140 of the respiratory therapy system, through the user interface conduit, and the connector 128, and into a volume of space define by the cushion 122 (or cushion 122 and frame 124) of the user interface 120 against a user's face. From the volume of space, the pressurized air reaches the user's airway through the user's mouth, nose, or both.

In some implementations, the connector 128 includes a plurality of vents 130 for permitting the escape of carbon dioxide (CO₂) and other gases exhaled by the user when the respiratory therapy device is active. In such implementations, each of the plurality of vents 130 is an opening that may be angled relative to the thickness of the connector wall through which the opening is formed. The angled openings can reduce noise of the CO₂ and other gases escaping to the atmosphere. Because of the reduced noise, acoustic signal associated with the plurality of vents 130 may be more apparent to an internal microphone, as opposed to an external microphone. Thus, an internal microphone may be located within, or otherwise physically integrated with, the respiratory therapy system and in acoustic communication with the flow of air which, in operation, is generated by the flow generator of the respiratory therapy device, and passes through the conduit and to the user interface 120.

In some implementations, the connector 128 optionally includes at least one valve 130 for permitting the escape of CO₂ and other gases exhaled by the user when the respiratory therapy device is inactive. In some implementations, the valve 130 (an example of an anti-asphyxia valve) includes a silicone (or other suitable material) flap that is a failsafe component, which allows CO₂ and other gases exhaled by the user to escape in the event that the vents 130 fail when the respiratory therapy device is active. In such implementations, when the silicone flap is open, the valve opening is much greater than each vent opening, and therefore less likely to be blocked by occlusion materials.

In some cases, the user interface 120 can be an indirect headgear user interface 120 and can include headgear 126, a cushion 122, and a connector 128. The headgear 126 includes strap and a headgear conduit. The headgear 126 is configured to be positioned generally about at least a portion of a user's head when the user wears the user interface 120. The headgear 126 includes a strap that can be coupled to the headgear conduit and positioned on the user's head such that the user's head is positioned between the strap and the headgear conduit. The cushion 122 is positioned between the user's face and the headgear conduit to form a seal on the user's face.

In such cases, the connector 128 can be configured to couple to the headgear 126 at one end and a conduit 140 of the respiratory therapy system 100 at the other end. In other implementations, the connector 128 is not included and the headgear 126 can alternatively connect directly to conduit 140 of the respiratory therapy system 100. The headgear conduit can be configured to deliver pressurized air from the conduit 140 of the respiratory therapy system 100 to the cushion 122, or more specifically, to the volume of space around the mouth and/or nose of the user and enclosed by the user cushion 122. The headgear conduit is hollow to provide a passageway for the pressurized air. Both sides of the headgear conduit can be hollow to provide two passageways for the pressurized air. Alternatively, only one side of the headgear conduit can be hollow to provide a single passageway. In some cases, headgear conduit comprises two passageways which, in use, are positioned at either side of a user's head/face. Alternatively, only one passageway of the headgear conduit can be hollow to provide a single passageway. The pressurized air can flow from the conduit 140 of the respiratory therapy system 100, through the connector 128 and the headgear conduit, and into the volume of space between the cushion 122 and the user's face. From the volume of space between the cushion 122 and the user's face, the pressurized air reaches the user's airway through the user's mouth, nose, or both.

In some implementations, the cushion 122 includes a plurality of vents 130 on the cushion 122 itself. Additionally, or alternatively, in some implementations, the connector 128 includes a plurality of vents 130 (“diffuser vents”) in proximity to the headgear 126, for permitting the escape of carbon dioxide (CO₂) and other gases exhaled by the user when the respiratory therapy device is active. In some implementations, the headgear 126 may include at least one plus anti-asphyxia valve (AAV) in proximity to the cushion 122, which allows CO₂ and other gases exhaled by the user to escape in the event that the vents 130 fail when the respiratory therapy device is active.

The conduit 140 (also referred to as an air circuit or tube) allows the flow of air between components of the respiratory therapy system 100, such as between the respiratory therapy device 110 and the user interface 120. In some implementations, there can be separate limbs of the conduit for inhalation and exhalation. In other implementations, a single limb conduit is used for both inhalation and exhalation.

The conduit 140 can include a first end that is coupled to the air outlet 118 of the respiratory therapy device 110. The first end can be coupled to the air outlet 118 of the respiratory therapy device 110 using a variety of techniques (e.g., a press fit connection, a snap fit connection, a threaded connection, etc.). In some implementations, the conduit 140 includes one or more heating elements that heat the pressurized air flowing through the conduit 140 (e.g., heat the air to a predetermined temperature or within a range of predetermined temperatures). Such heating elements can be coupled to and/or imbedded in the conduit 140. In such implementations, the first end can include an electrical contact that is electrically coupled to the respiratory therapy device 110 to power the one or more heating elements of the conduit 140. For example, the electrical contact can be electrically coupled to an electrical contact of the air outlet 118 of the respiratory therapy device 110. In this example, electrical contact of the conduit 140 can be a male connector and the electrical contact of the air outlet 118 can be female connector, or, alternatively, the opposite configuration can be used.

The display device 150 is generally used to display image(s) including still images, video images, or both and/or information regarding the respiratory therapy device 110. For example, the display device 150 can provide information regarding the status of the respiratory therapy device 110 (e.g., whether the respiratory therapy device 110 is on/off, the pressure of the air being delivered by the respiratory therapy device 110, the temperature of the air being delivered by the respiratory therapy device 110, etc.) and/or other information (e.g., a sleep score and/or a therapy score, also referred to as a my Air™ score, such as described in WO 2016/061629 and U.S. Patent Pub. No. 2017/0311879, which are hereby incorporated by reference herein in their entireties, the current date/time, personal information for the user 20, etc.). In some cases, one or more avatars or related information or messages can be presented via display device 150. In some implementations, the display device 150 acts as a human-machine interface (HMI) that includes a graphic user interface (GUI) configured to display the image(s) as an input interface. The display device 150 can be an LED display, an OLED display, an LCD display, or the like. The input interface can be, for example, a touchscreen or touch-sensitive substrate, a mouse, a keyboard, or any sensor system configured to sense inputs made by a human user interacting with the respiratory therapy device 110.

The humidifier 160 is coupled to or integrated in the respiratory therapy device 110 and includes a reservoir 162 for storing water that can be used to humidify the pressurized air delivered from the respiratory therapy device 110. The humidifier 160 includes a one or more heating elements 164 to heat the water in the reservoir to generate water vapor. The humidifier 160 can be fluidly coupled to a water vapor inlet of the air pathway between the blower motor 114 and the air outlet 118, or can be formed in-line with the air pathway between the blower motor 114 and the air outlet 118. In an example, air can flow from an air inlet 116 through the blower motor 114, and then through the humidifier 160 before exiting the respiratory therapy device 110 via the air outlet 118.

While the respiratory therapy system 100 has been described herein as including each of the respiratory therapy device 110, the user interface 120, the conduit 140, the display device 150, and the humidifier 160, more or fewer components can be included in a respiratory therapy system according to implementations of the present disclosure. For example, a first alternative respiratory therapy system includes the respiratory therapy device 110, the user interface 120, and the conduit 140. As another example, a second alternative system includes the respiratory therapy device 110, the user interface 120, and the conduit 140, and the display device 150. Thus, various respiratory therapy systems can be formed using any portion or portions of the components shown and described herein and/or in combination with one or more other components.

The control system 200 includes one or more processors 202 (hereinafter, processor 202). The control system 200 is generally used to control (e.g., actuate) the various components of the system 10 and/or analyze data obtained and/or generated by the components of the system 10. The processor 202 can be a general or special purpose processor or microprocessor. While one processor 202 is illustrated in FIG. 1, the control system 200 can include any number of processors (e.g., one processor, two processors, five processors, ten processors, etc.) that can be in a single housing, or located remotely from each other. The control system 200 (or any other control system) or a portion of the control system 200 such as the processor 202 (or any other processor(s) or portion(s) of any other control system), can be used to carry out one or more steps of any of the methods described and/or claimed herein. The control system 200 can be coupled to and/or positioned within, for example, a housing of the user device 260, a portion (e.g., the respiratory therapy device 110) of the respiratory therapy system 100, and/or within a housing of one or more of the sensors 210. The control system 200 can be centralized (within one such housing) or decentralized (within two or more of such housings, which are physically distinct). In such implementations including two or more housings containing the control system 200, the housings can be located proximately and/or remotely from each other.

The memory device 204 stores machine-readable instructions that are executable by the processor 202 of the control system 200. The memory device 204 can be any suitable computer readable storage device or media, such as, for example, a random or serial access memory device, a hard drive, a solid state drive, a flash memory device, etc. While one memory device 204 is shown in FIG. 1, the system 10 can include any suitable number of memory devices 204 (e.g., one memory device, two memory devices, five memory devices, ten memory devices, etc.). The memory device 204 can be coupled to and/or positioned within a housing of a respiratory therapy device 110 of the respiratory therapy system 100, within a housing of the user device 260, within a housing of one or more of the sensors 210, or any combination thereof. Like the control system 200, the memory device 204 can be centralized (within one such housing) or decentralized (within two or more of such housings, which are physically distinct).

In some implementations, the memory device 204 stores a user profile associated with the user. The user profile can include, for example, demographic information associated with the user, biometric information associated with the user, medical information associated with the user, self-reported user feedback, sleep parameters associated with the user (e.g., sleep-related parameters recorded from one or more earlier sleep sessions), or any combination thereof. The demographic information can include, for example, information indicative of an age of the user, a gender of the user, a race of the user, a geographic location of the user, a relationship status, a family history of insomnia or sleep apnea, an employment status of the user, an educational status of the user, a socioeconomic status of the user, or any combination thereof. The medical information can include, for example, information indicative of one or more medical conditions associated with the user, medication usage by the user, or both. The medical information data can further include a multiple sleep latency test (MSLT) result or score and/or a Pittsburgh Sleep Quality Index (PSQI) score or value. The self-reported user feedback can include information indicative of a self-reported subjective sleep score (e.g., poor, average, excellent), a self-reported subjective stress level of the user, a self-reported subjective fatigue level of the user, a self-reported subjective health status of the user, a recent life event experienced by the user, or any combination thereof.

In some cases, the memory device 204 can store avatar information, such as a model used to generate a digital avatar; one or more parameters used to generate a digital avatar from a model; milestone information; achievement information; etc.

As described herein, the processor 202 and/or memory device 204 can receive data (e.g., physiological data and/or audio data) from the one or more sensors 210 such that the data for storage in the memory device 204 and/or for analysis by the processor 202. The processor 202 and/or memory device 204 can communicate with the one or more sensors 210 using a wired connection or a wireless connection (e.g., using an RF communication protocol, a Wi-Fi communication protocol, a Bluetooth communication protocol, over a cellular network, etc.). In some implementations, the system 10 can include an antenna, a receiver (e.g., an RF receiver), a transmitter (e.g., an RF transmitter), a transceiver, or any combination thereof. Such components can be coupled to or integrated a housing of the control system 200 (e.g., in the same housing as the processor 202 and/or memory device 204), or the user device 260.

The one or more sensors 210 include a pressure sensor 212, a flow rate sensor 214, temperature sensor 216, a motion sensor 218, a microphone 220, a speaker 222, a radio-frequency (RF) receiver 226, a RF transmitter 228, a camera 232, an infrared sensor 234, a photoplethysmogram (PPG) sensor 236, an electrocardiogram (ECG) sensor 238, an electroencephalography (EEG) sensor 240, a capacitive sensor 242, a force sensor 244, a strain gauge sensor 246, an electromyography (EMG) sensor 248, an oxygen sensor 250, an analyte sensor 252, a moisture sensor 254, a LiDAR sensor 256, or any combination thereof. Generally, each of the one or more sensors 210 are configured to output sensor data that is received and stored in the memory device 204 or one or more other memory devices.

While the one or more sensors 210 are shown and described as including each of the pressure sensor 212, the flow rate sensor 214, the temperature sensor 216, the motion sensor 218, the microphone 220, the speaker 222, the RF receiver 226, the RF transmitter 228, the camera 232, the infrared sensor 234, the photoplethysmogram (PPG) sensor 236, the electrocardiogram (ECG) sensor 238, the electroencephalography (EEG) sensor 240, the capacitive sensor 242, the force sensor 244, the strain gauge sensor 246, the electromyography (EMG) sensor 248, the oxygen sensor 250, the analyte sensor 252, the moisture sensor 254, and the LiDAR sensor 256, more generally, the one or more sensors 210 can include any combination and any number of each of the sensors described and/or shown herein.

As described herein, the system 10 generally can be used to generate physiological data associated with a user (e.g., a user of the respiratory therapy system 100) during a sleep session. The physiological data can be analyzed to generate one or more sleep-related parameters, which can include any parameter, measurement, etc. related to the user during the sleep session. The one or more sleep-related parameters that can be determined for the user 20 during the sleep session include, for example, an Apnea-Hypopnea Index (AHI) score, a sleep score, a flow signal, a respiration signal, a respiration rate, an inspiration amplitude, an expiration amplitude, an inspiration-expiration ratio, a number of events per hour, a pattern of events, a stage, pressure settings of the respiratory therapy device 110, a heart rate, a heart rate variability, movement of the user 20, temperature, EEG activity, EMG activity, arousal, snoring, choking, coughing, whistling, wheezing, or any combination thereof.

The one or more sensors 210 can be used to generate, for example, physiological data, audio data, or both. Physiological data generated by one or more of the sensors 210 can be used by the control system 200 to determine a sleep-wake signal associated with the user 20 (FIG. 2) during the sleep session and one or more sleep-related parameters. The sleep-wake signal can be indicative of one or more sleep states, including wakefulness, relaxed wakefulness, micro-awakenings, or distinct sleep stages such as, for example, a rapid eye movement (REM) stage, a first non-REM stage (often referred to as “N1”), a second non-REM stage (often referred to as “N2”), a third non-REM stage (often referred to as “N3”), or any combination thereof. Methods for determining sleep states and/or sleep stages from physiological data generated by one or more sensors, such as the one or more sensors 210, are described in, for example, WO 2014/047310, U.S. Patent Pub. No. 2014/0088373, WO 2017/132726, WO 2019/122413, WO 2019/122414, and U.S. Patent Pub. No. 2020/0383580 each of which is hereby incorporated by reference herein in its entirety.

In some implementations, the sleep-wake signal described herein can be timestamped to indicate a time that the user enters the bed, a time that the user exits the bed, a time that the user attempts to fall asleep, etc. The sleep-wake signal can be measured by the one or more sensors 210 during the sleep session at a predetermined sampling rate, such as, for example, one sample per second, one sample per 30 seconds, one sample per minute, etc. In some implementations, the sleep-wake signal can also be indicative of a respiration signal, a respiration rate, an inspiration amplitude, an expiration amplitude, an inspiration-expiration ratio, a number of events per hour, a pattern of events, pressure settings of the respiratory therapy device 110, or any combination thereof during the sleep session. The event(s) can include snoring, apneas, central apneas, obstructive apneas, mixed apneas, hypopneas, a mask leak (e.g., from the user interface 120), a restless leg, a sleeping disorder, choking, an increased heart rate, labored breathing, an asthma attack, an epileptic episode, a seizure, or any combination thereof. The one or more sleep-related parameters that can be determined for the user during the sleep session based on the sleep-wake signal include, for example, a total time in bed, a total sleep time, a sleep onset latency, a wake-after-sleep-onset parameter, a sleep efficiency, a fragmentation index, or any combination thereof. As described in further detail herein, the physiological data and/or the sleep-related parameters can be analyzed to determine one or more sleep-related scores.

Physiological data and/or audio data generated by the one or more sensors 210 can also be used to determine a respiration signal associated with a user during a sleep session. The respiration signal is generally indicative of respiration or breathing of the user during the sleep session. The respiration signal can be indicative of and/or analyzed to determine (e.g., using the control system 200) one or more sleep-related parameters, such as, for example, a respiration rate, a respiration rate variability, an inspiration amplitude, an expiration amplitude, an inspiration-expiration ratio, an occurrence of one or more events, a number of events per hour, a pattern of events, a sleep state, a sleet stage, an apnea-hypopnea index (AHI), pressure settings of the respiratory therapy device 110, or any combination thereof. The one or more events can include snoring, apneas, central apneas, obstructive apneas, mixed apneas, hypopneas, a mask leak (e.g., from the user interface 120), a cough, a restless leg, a sleeping disorder, choking, an increased heart rate, labored breathing, an asthma attack, an epileptic episode, a seizure, increased blood pressure, or any combination thereof. Many of the described sleep-related parameters are physiological parameters, although some of the sleep-related parameters can be considered to be non-physiological parameters. Other types of physiological and/or non-physiological parameters can also be determined, either from the data from the one or more sensors 210, or from other types of data.

The pressure sensor 212 outputs pressure data that can be stored in the memory device 204 and/or analyzed by the processor 202 of the control system 200. In some implementations, the pressure sensor 212 is an air pressure sensor (e.g., barometric pressure sensor) that generates sensor data indicative of the respiration (e.g., inhaling and/or exhaling) of the user of the respiratory therapy system 100 and/or ambient pressure. In such implementations, the pressure sensor 212 can be coupled to or integrated in the respiratory therapy device 110. The pressure sensor 212 can be, for example, a capacitive sensor, an electromagnetic sensor, a piezoelectric sensor, a strain-gauge sensor, an optical sensor, a potentiometric sensor, or any combination thereof.

The flow rate sensor 214 outputs flow rate data that can be stored in the memory device 204 and/or analyzed by the processor 202 of the control system 200. Examples of flow rate sensors (such as, for example, the flow rate sensor 214) are described in International Publication No. WO 2012/012835 and U.S. Pat. No. 10,328,219, both of which are hereby incorporated by reference herein in their entireties. In some implementations, the flow rate sensor 214 is used to determine an air flow rate from the respiratory therapy device 110, an air flow rate through the conduit 140, an air flow rate through the user interface 120, or any combination thereof. In such implementations, the flow rate sensor 214 can be coupled to or integrated in the respiratory therapy device 110, the user interface 120, or the conduit 140. The flow rate sensor 214 can be a mass flow rate sensor such as, for example, a rotary flow meter (e.g., Hall effect flow meters), a turbine flow meter, an orifice flow meter, an ultrasonic flow meter, a hot wire sensor, a vortex sensor, a membrane sensor, or any combination thereof. In some implementations, the flow rate sensor 214 is configured to measure a vent flow (e.g., intentional “leak”), an unintentional leak (e.g., mouth leak and/or mask leak), a patient flow (e.g., air into and/or out of lungs), or any combination thereof. In some implementations, the flow rate data can be analyzed to determine cardiogenic oscillations of the user. In some examples, the pressure sensor 212 can be used to determine a blood pressure of a user.

The temperature sensor 216 outputs temperature data that can be stored in the memory device 204 and/or analyzed by the processor 202 of the control system 200. In some implementations, the temperature sensor 216 generates temperatures data indicative of a core body temperature of the user 20 (FIG. 2), a skin temperature of the user 20, a temperature of the air flowing from the respiratory therapy device 110 and/or through the conduit 140, a temperature in the user interface 120, an ambient temperature, or any combination thereof. The temperature sensor 216 can be, for example, a thermocouple sensor, a thermistor sensor, a silicon band gap temperature sensor or semiconductor-based sensor, a resistance temperature detector, or any combination thereof.

The motion sensor 218 outputs motion data that can be stored in the memory device 204 and/or analyzed by the processor 202 of the control system 200. The motion sensor 218 can be used to detect movement of the user 20 during the sleep session, and/or detect movement of any of the components of the respiratory therapy system 100, such as the respiratory therapy device 110, the user interface 120, or the conduit 140. The motion sensor 218 can include one or more inertial sensors, such as accelerometers, gyroscopes, and magnetometers. In some implementations, the motion sensor 218 alternatively or additionally generates one or more signals representing bodily movement of the user, from which may be obtained a signal representing a sleep state of the user; for example, via a respiratory movement of the user. In some implementations, the motion data from the motion sensor 218 can be used in conjunction with additional data from another one of the sensors 210 to determine the sleep state of the user.

The microphone 220 outputs sound and/or audio data that can be stored in the memory device 204 and/or analyzed by the processor 202 of the control system 200. The audio data generated by the microphone 220 is reproducible as one or more sound(s) during a sleep session (e.g., sounds from the user 20). The audio data form the microphone 220 can also be used to identify (e.g., using the control system 200) an event experienced by the user during the sleep session, as described in further detail herein. The microphone 220 can be coupled to or integrated in the respiratory therapy device 110, the user interface 120, the conduit 140, or the user device 260. In some implementations, the system 10 includes a plurality of microphones (e.g., two or more microphones and/or an array of microphones with beamforming) such that sound data generated by each of the plurality of microphones can be used to discriminate the sound data generated by another of the plurality of microphones The speaker 222 outputs sound waves that are audible to a user of the system 10 (e.g., the user 20 of FIG. 2). The speaker 222 can be used, for example, as an alarm clock or to play an alert or message to the user 20 (e.g., in response to an event). In some implementations, the speaker 222 can be used to communicate the audio data generated by the microphone 220 to the user. The speaker 222 can be coupled to or integrated in the respiratory therapy device 110, the user interface 120, the conduit 140, or the user device 260.

The microphone 220 and the speaker 222 can be used as separate devices. In some implementations, the microphone 220 and the speaker 222 can be combined into an acoustic sensor 224 (e.g., a SONAR sensor), as described in, for example, WO 2018/050913, WO 2020/104465, U.S. Pat. App. Pub. No. 2022/0007965, each of which is hereby incorporated by reference herein in its entirety. In such implementations, the speaker 222 generates or emits sound waves at a predetermined interval and the microphone 220 detects the reflections of the emitted sound waves from the speaker 222. The sound waves generated or emitted by the speaker 222 have a frequency that is not audible to the human ear (e.g., below 20 Hz or above around 18 kHz) so as not to disturb the sleep of the user 20 or the bed partner 30 (FIG. 2). Based at least in part on the data from the microphone 220 and/or the speaker 222, the control system 200 can determine a location of the user 20 (FIG. 2) and/or one or more of the sleep-related parameters described in herein such as, for example, a respiration signal, a respiration rate, an inspiration amplitude, an expiration amplitude, an inspiration-expiration ratio, a number of events per hour, a pattern of events, a sleep state, a sleep stage, pressure settings of the respiratory therapy device 110, or any combination thereof. In such a context, a sonar sensor may be understood to concern an active acoustic sensing, such as by generating and/or transmitting ultrasound and/or low frequency ultrasound sensing signals (e.g., in a frequency range of about 17-23 kHz, 18-22 kHz, or 17-18 kHz, for example), through the air.

In some implementations, the sensors 210 include (i) a first microphone that is the same as, or similar to, the microphone 220, and is integrated in the acoustic sensor 224 and (ii) a second microphone that is the same as, or similar to, the microphone 220, but is separate and distinct from the first microphone that is integrated in the acoustic sensor 224.

The RF transmitter 228 generates and/or emits radio waves having a predetermined frequency and/or a predetermined amplitude (e.g., within a high frequency band, within a low frequency band, long wave signals, short wave signals, etc.). The RF receiver 226 detects the reflections of the radio waves emitted from the RF transmitter 228, and this data can be analyzed by the control system 200 to determine a location of the user and/or one or more of the sleep-related parameters described herein. An RF receiver (either the RF receiver 226 and the RF transmitter 228 or another RF pair) can also be used for wireless communication between the control system 200, the respiratory therapy device 110, the one or more sensors 210, the user device 260, or any combination thereof. While the RF receiver 226 and RF transmitter 228 are shown as being separate and distinct elements in FIG. 1, in some implementations, the RF receiver 226 and RF transmitter 228 are combined as a part of an RF sensor 230 (e.g. a RADAR sensor). In some such implementations, the RF sensor 230 includes a control circuit. The format of the RF communication can be Wi-Fi, Bluetooth, or the like.

In some implementations, the RF sensor 230 is a part of a mesh system. One example of a mesh system is a Wi-Fi mesh system, which can include mesh nodes, mesh router(s), and mesh gateway(s), each of which can be mobile/movable or fixed. In such implementations, the Wi-Fi mesh system includes a Wi-Fi router and/or a Wi-Fi controller and one or more satellites (e.g., access points), each of which include an RF sensor that the is the same as, or similar to, the RF sensor 230. The Wi-Fi router and satellites continuously communicate with one another using Wi-Fi signals. The Wi-Fi mesh system can be used to generate motion data based on changes in the Wi-Fi signals (e.g., differences in received signal strength) between the router and the satellite(s) due to an object or person moving partially obstructing the signals. The motion data can be indicative of motion, breathing, heart rate, gait, falls, behavior, etc., or any combination thereof.

The camera 232 outputs image data reproducible as one or more images (e.g., still images, video images, thermal images, or any combination thereof) that can be stored in the memory device 204. The image data from the camera 232 can be used by the control system 200 to determine one or more of the sleep-related parameters described herein, such as, for example, one or more events (e.g., periodic limb movement or restless leg syndrome), a respiration signal, a respiration rate, an inspiration amplitude, an expiration amplitude, an inspiration-expiration ratio, a number of events per hour, a pattern of events, a sleep state, a sleep stage, or any combination thereof. Further, the image data from the camera 232 can be used to, for example, identify a location of the user, to determine chest movement of the user (FIG. 2), to determine air flow of the mouth and/or nose of the user, to determine a time when the user enters the bed (FIG. 2), and to determine a time when the user exits the bed. In some implementations, the camera 232 includes a wide angle lens or a fish eye lens.

The infrared (IR) sensor 234 outputs infrared image data reproducible as one or more infrared images (e.g., still images, video images, or both) that can be stored in the memory device 204. The infrared data from the IR sensor 234 can be used to determine one or more sleep-related parameters during a sleep session, including a temperature of the user 20 and/or movement of the user 20. The IR sensor 234 can also be used in conjunction with the camera 232 when measuring the presence, location, and/or movement of the user 20. The IR sensor 234 can detect infrared light having a wavelength between about 700 nm and about 1 mm, for example, while the camera 232 can detect visible light having a wavelength between about 380 nm and about 740 nm.

The PPG sensor 236 outputs physiological data associated with the user 20 (FIG. 2) that can be used to determine one or more sleep-related parameters, such as, for example, a heart rate, a heart rate variability, a cardiac cycle, respiration rate, an inspiration amplitude, an expiration amplitude, an inspiration-expiration ratio, estimated blood pressure parameter(s), or any combination thereof. The PPG sensor 236 can be worn by the user 20, embedded in clothing and/or fabric that is worn by the user 20, embedded in and/or coupled to the user interface 120 and/or its associated headgear (e.g., straps, etc.), etc.

The ECG sensor 238 outputs physiological data associated with electrical activity of the heart of the user 20. In some implementations, the ECG sensor 238 includes one or more electrodes that are positioned on or around a portion of the user 20 during the sleep session. The physiological data from the ECG sensor 238 can be used, for example, to determine one or more of the sleep-related parameters described herein.

The EEG sensor 240 outputs physiological data associated with electrical activity of the brain of the user 20. In some implementations, the EEG sensor 240 includes one or more electrodes that are positioned on or around the scalp of the user 20 during the sleep session. The physiological data from the EEG sensor 240 can be used, for example, to determine a sleep state and/or a sleep stage of the user 20 at any given time during the sleep session. In some implementations, the EEG sensor 240 can be integrated in the user interface 120 and/or the associated headgear (e.g., straps, etc.).

The capacitive sensor 242, the force sensor 244, and the strain gauge sensor 246 output data that can be stored in the memory device 204 and used/analyzed by the control system 200 to determine, for example, one or more of the sleep-related parameters described herein. The EMG sensor 248 outputs physiological data associated with electrical activity produced by one or more muscles. The oxygen sensor 250 outputs oxygen data indicative of an oxygen concentration of gas (e.g., in the conduit 140 or at the user interface 120). The oxygen sensor 250 can be, for example, an ultrasonic oxygen sensor, an electrical oxygen sensor, a chemical oxygen sensor, an optical oxygen sensor, a pulse oximeter (e.g., SpO₂ sensor), or any combination thereof.

The analyte sensor 252 can be used to detect the presence of an analyte in the exhaled breath of the user 20. The data output by the analyte sensor 252 can be stored in the memory device 204 and used by the control system 200 to determine the identity and concentration of any analytes in the breath of the user. In some implementations, the analyte sensor 174 is positioned near a mouth of the user to detect analytes in breath exhaled from the user's mouth. For example, when the user interface 120 is a facial mask that covers the nose and mouth of the user, the analyte sensor 252 can be positioned within the facial mask to monitor the user's mouth breathing. In other implementations, such as when the user interface 120 is a nasal mask or a nasal pillow mask, the analyte sensor 252 can be positioned near the nose of the user to detect analytes in breath exhaled through the user's nose. In still other implementations, the analyte sensor 252 can be positioned near the user's mouth when the user interface 120 is a nasal mask or a nasal pillow mask. In this implementation, the analyte sensor 252 can be used to detect whether any air is inadvertently leaking from the user's mouth and/or the user interface 120. In some implementations, the analyte sensor 252 is a volatile organic compound (VOC) sensor that can be used to detect carbon-based chemicals or compounds. In some implementations, the analyte sensor 174 can also be used to detect whether the user is breathing through their nose or mouth. For example, if the data output by an analyte sensor 252 positioned near the mouth of the user or within the facial mask (e.g., in implementations where the user interface 120 is a facial mask) detects the presence of an analyte, the control system 200 can use this data as an indication that the user is breathing through their mouth.

The moisture sensor 254 outputs data that can be stored in the memory device 204 and used by the control system 200. The moisture sensor 254 can be used to detect moisture in various areas surrounding the user (e.g., inside the conduit 140 or the user interface 120, near the user's face, near the connection between the conduit 140 and the user interface 120, near the connection between the conduit 140 and the respiratory therapy device 110, etc.). Thus, in some implementations, the moisture sensor 254 can be coupled to or integrated in the user interface 120 or in the conduit 140 to monitor the humidity of the pressurized air from the respiratory therapy device 110. In other implementations, the moisture sensor 254 is placed near any area where moisture levels need to be monitored. The moisture sensor 254 can also be used to monitor the humidity of the ambient environment surrounding the user, for example, the air inside the bedroom.

The Light Detection and Ranging (LiDAR) sensor 256 can be used for depth sensing. This type of optical sensor (e.g., laser sensor) can be used to detect objects and build three dimensional (3D) maps of the surroundings, such as of a living space. LiDAR can generally utilize a pulsed laser to make time of flight measurements. LiDAR is also referred to as 3D laser scanning. In an example of use of such a sensor, a fixed or mobile device (such as a smartphone) having a LiDAR sensor 256 can measure and map an area extending 5 meters or more away from the sensor. The LiDAR data can be fused with point cloud data estimated by an electromagnetic RADAR sensor, for example. The LiDAR sensor(s) 256 can also use artificial intelligence (AI) to automatically geofence RADAR systems by detecting and classifying features in a space that might cause issues for RADAR systems, such a glass windows (which can be highly reflective to RADAR). LiDAR can also be used to provide an estimate of the height of a person, as well as changes in height when the person sits down, or falls down, for example. LiDAR may be used to form a 3D mesh representation of an environment. In a further use, for solid surfaces through which radio waves pass (e.g., radio-translucent materials), the LiDAR may reflect off such surfaces, thus allowing a classification of different type of obstacles.

In some implementations, the one or more sensors 210 also include a galvanic skin response (GSR) sensor, a blood flow sensor, a respiration sensor, a pulse sensor, a sphygmomanometer sensor, an oximetry sensor, a sonar sensor, a RADAR sensor, a blood glucose sensor, a color sensor, a pH sensor, an air quality sensor, a tilt sensor, a rain sensor, a soil moisture sensor, a water flow sensor, an alcohol sensor, or any combination thereof.

While shown separately in FIG. 1, any combination of the one or more sensors 210 can be integrated in and/or coupled to any one or more of the components of the system 100, including the respiratory therapy device 110, the user interface 120, the conduit 140, the humidifier 160, the control system 200, the user device 260, the activity tracker 270, or any combination thereof. For example, the microphone 220 and the speaker 222 can be integrated in and/or coupled to the user device 260 and the pressure sensor 212 and/or flow rate sensor 132 are integrated in and/or coupled to the respiratory therapy device 110. In some implementations, at least one of the one or more sensors 210 is not coupled to the respiratory therapy device 110, the control system 200, or the user device 260, and is positioned generally adjacent to the user 20 during the sleep session (e.g., positioned on or in contact with a portion of the user 20, worn by the user 20, coupled to or positioned on the nightstand, coupled to the mattress, coupled to the ceiling, etc.).

One or more of the respiratory therapy device 110, the user interface 120, the conduit 140, the display device 150, and the humidifier 160 can contain one or more sensors (e.g., a pressure sensor, a flow rate sensor, or more generally any of the other sensors 210 described herein). These one or more sensors can be used, for example, to measure the air pressure and/or flow rate of pressurized air supplied by the respiratory therapy device 110.

The data from the one or more sensors 210 can be analyzed (e.g., by the control system 200) to determine one or more sleep-related parameters, which can include a respiration signal, a respiration rate, a respiration pattern, an inspiration amplitude, an expiration amplitude, an inspiration-expiration ratio, an occurrence of one or more events, a number of events per hour, a pattern of events, a sleep state, an apnea-hypopnea index (AHI), or any combination thereof. The one or more events can include snoring, apneas, central apneas, obstructive apneas, mixed apneas, hypopneas, a mask leak, a cough, a restless leg, a sleeping disorder, choking, an increased heart rate, labored breathing, an asthma attack, an epileptic episode, a seizure, increased blood pressure, or any combination thereof. Many of these sleep-related parameters are physiological parameters, although some of the sleep-related parameters can be considered to be non-physiological parameters. Other types of physiological and non-physiological parameters can also be determined, either from the data from the one or more sensors 210, or from other types of data.

The user device 260 (FIG. 1) includes a display device 262. In some cases, one or more avatars or related information or messages can be presented via display device 262. The user device 260 can be, for example, a mobile device such as a smart phone, a tablet, a gaming console, a smart watch, a laptop, or the like. Alternatively, the user device 260 can be an external sensing system, a television (e.g., a smart television) or another smart home device (e.g., a smart speaker(s) such as Google Home, Amazon Echo, Alexa etc.). In some implementations, the user device is a wearable device (e.g., a smart watch). The display device 262 is generally used to display image(s) including still images, video images, or both. In some implementations, the display device 262 acts as a human-machine interface (HMI) that includes a graphic user interface (GUI) configured to display the image(s) and an input interface. The display device 262 can be an LED display, an OLED display, an LCD display, or the like. The input interface can be, for example, a touchscreen or touch-sensitive substrate, a mouse, a keyboard, or any sensor system configured to sense inputs made by a human user interacting with the user device 260. In some implementations, one or more user devices can be used by and/or included in the system 10.

In some implementations, the system 100 also includes an activity tracker 270. The activity tracker 270 is generally used to aid in generating physiological data associated with the user. The activity tracker 270 can include one or more of the sensors 210 described herein, such as, for example, the motion sensor 138 (e.g., one or more accelerometers and/or gyroscopes), the PPG sensor 154, and/or the ECG sensor 156. The physiological data from the activity tracker 270 can be used to determine, for example, a number of steps, a distance traveled, a number of steps climbed, a duration of physical activity, a type of physical activity, an intensity of physical activity, time spent standing, a respiration rate, an average respiration rate, a resting respiration rate, a maximum he respiration art rate, a respiration rate variability, a heart rate, an average heart rate, a resting heart rate, a maximum heart rate, a heart rate variability, a number of calories burned, blood oxygen saturation, electrodermal activity (also known as skin conductance or galvanic skin response), or any combination thereof. In some implementations, the activity tracker 270 is coupled (e.g., electronically or physically) to the user device 260.

In some implementations, the activity tracker 270 is a wearable device that can be worn by the user, such as a smartwatch, a wristband, a ring, or a patch. For example, referring to FIG. 2, the activity tracker 270 is worn on a wrist of the user 20. The activity tracker 270 can also be coupled to or integrated a garment or clothing that is worn by the user. Alternatively still, the activity tracker 270 can also be coupled to or integrated in (e.g., within the same housing) the user device 260. More generally, the activity tracker 270 can be communicatively coupled with, or physically integrated in (e.g., within a housing), the control system 200, the memory device 204, the respiratory therapy system 100, and/or the user device 260.

In some implementations, the system 100 also includes a blood pressure device 280. The blood pressure device 280 is generally used to aid in generating cardiovascular data for determining one or more blood pressure measurements associated with the user 20. The blood pressure device 280 can include at least one of the one or more sensors 210 to measure, for example, a systolic blood pressure component and/or a diastolic blood pressure component.

In some implementations, the blood pressure device 280 is a sphygmomanometer including an inflatable cuff that can be worn by the user 20 and a pressure sensor (e.g., the pressure sensor 212 described herein). For example, in the example of FIG. 2, the blood pressure device 280 can be worn on an upper arm of the user 20. In such implementations where the blood pressure device 280 is a sphygmomanometer, the blood pressure device 280 also includes a pump (e.g., a manually operated bulb) for inflating the cuff. In some implementations, the blood pressure device 280 is coupled to the respiratory therapy device 110 of the respiratory therapy system 100, which in turn delivers pressurized air to inflate the cuff. More generally, the blood pressure device 280 can be communicatively coupled with, and/or physically integrated in (e.g., within a housing), the control system 200, the memory device 204, the respiratory therapy system 100, the user device 260, and/or the activity tracker 270.

In other implementations, the blood pressure device 280 is an ambulatory blood pressure monitor communicatively coupled to the respiratory therapy system 100. An ambulatory blood pressure monitor includes a portable recording device attached to a belt or strap worn by the user 20 and an inflatable cuff attached to the portable recording device and worn around an arm of the user 20. The ambulatory blood pressure monitor is configured to measure blood pressure between about every fifteen minutes to about thirty minutes over a 24-hour or a 48-hour period. The ambulatory blood pressure monitor may measure heart rate of the user 20 at the same time. These multiple readings are averaged over the 24-hour period. The ambulatory blood pressure monitor determines any changes in the measured blood pressure and heart rate of the user 20, as well as any distribution and/or trending patterns of the blood pressure and heart rate data during a sleeping period and an awakened period of the user 20. The measured data and statistics may then be communicated to the respiratory therapy system 100.

The blood pressure device 280 maybe positioned external to the respiratory therapy system 100, coupled directly or indirectly to the user interface 120, coupled directly or indirectly to a headgear associated with the user interface 120, or inflatably coupled to or about a portion of the user 20. The blood pressure device 280 is generally used to aid in generating physiological data for determining one or more blood pressure measurements associated with a user, for example, a systolic blood pressure component and/or a diastolic blood pressure component. In some implementations, the blood pressure device 280 is a sphygmomanometer including an inflatable cuff that can be worn by a user and a pressure sensor (e.g., the pressure sensor 212 described herein).

In some implementations, the blood pressure device 280 is an invasive device which can continuously monitor arterial blood pressure of the user 20 and take an arterial blood sample on demand for analyzing gas of the arterial blood. In some other implementations, the blood pressure device 280 is a continuous blood pressure monitor, using a radio frequency sensor and capable of measuring blood pressure of the user 20 once very few seconds (e.g., every 3 seconds, every 5 seconds, every 7 seconds, etc.) The radio frequency sensor may use continuous wave, frequency-modulated continuous wave (FMCW with ramp chirp, triangle, sinewave), other schemes such as PSK, FSK etc., pulsed continuous wave, and/or spread in ultra wideband ranges (which may include spreading, PRN codes or impulse systems).

While the control system 200 and the memory device 204 are described and shown in FIG. 1 as being a separate and distinct component of the system 100, in some implementations, the control system 200 and/or the memory device 204 are integrated in the user device 260 and/or the respiratory therapy device 110. Alternatively, in some implementations, the control system 200 or a portion thereof (e.g., the processor 202) can be located in a cloud (e.g., integrated in a server, integrated in an Internet of Things (IoT) device, connected to the cloud, be subject to edge cloud processing, etc.), located in one or more servers (e.g., remote servers, local servers, etc., or any combination thereof.

While system 100 is shown as including all of the components described above, more or fewer components can be included in a system according to implementations of the present disclosure. For example, a first alternative system includes the control system 200, the memory device 204, and at least one of the one or more sensors 210 and does not include the respiratory therapy system 100. As another example, a second alternative system includes the control system 200, the memory device 204, at least one of the one or more sensors 210, and the user device 260. As yet another example, a third alternative system includes the control system 200, the memory device 204, the respiratory therapy system 100, at least one of the one or more sensors 210, and the user device 260. Thus, various systems can be formed using any portion or portions of the components shown and described herein and/or in combination with one or more other components.

As used herein, a sleep session can be defined in multiple ways. For example, a sleep session can be defined by an initial start time and an end time. In some implementations, a sleep session is a duration where the user is asleep, that is, the sleep session has a start time and an end time, and during the sleep session, the user does not wake until the end time. That is, any period of the user being awake is not included in a sleep session. From this first definition of sleep session, if the user wakes ups and falls asleep multiple times in the same night, each of the sleep intervals separated by an awake interval is a sleep session.

Alternatively, in some implementations, a sleep session has a start time and an end time, and during the sleep session, the user can wake up, without the sleep session ending, so long as a continuous duration that the user is awake is below an awake duration threshold. The awake duration threshold can be defined as a percentage of a sleep session. The awake duration threshold can be, for example, about twenty percent of the sleep session, about fifteen percent of the sleep session duration, about ten percent of the sleep session duration, about five percent of the sleep session duration, about two percent of the sleep session duration, etc., or any other threshold percentage. In some implementations, the awake duration threshold is defined as a fixed amount of time, such as, for example, about one hour, about thirty minutes, about fifteen minutes, about ten minutes, about five minutes, about two minutes, etc., or any other amount of time.

In some implementations, a sleep session is defined as the entire time between the time in the evening at which the user first entered the bed, and the time the next morning when user last left the bed. Put another way, a sleep session can be defined as a period of time that begins on a first date (e.g., Monday, Jan. 6, 2020) at a first time (e.g., 10:00 PM), that can be referred to as the current evening, when the user first enters a bed with the intention of going to sleep (e.g., not if the user intends to first watch television or play with a smart phone before going to sleep, etc.), and ends on a second date (e.g., Tuesday, Jan. 7, 2020) at a second time (e.g., 7:00 AM), that can be referred to as the next morning, when the user first exits the bed with the intention of not going back to sleep that next morning.

In some implementations, the user can manually define the beginning of a sleep session and/or manually terminate a sleep session. For example, the user can select (e.g., by clicking or tapping) one or more user-selectable element that is displayed on the display device 262 of the user device 260 (FIG. 1) to manually initiate or terminate the sleep session.

Generally, the sleep session includes any point in time after the user 20 has laid or sat down in the bed 40 (or another area or object on which they intend to sleep), and has turned on the respiratory therapy device 110 and donned the user interface 120. The sleep session can thus include time periods (i) when the user 20 is using the respiratory therapy system 100, but before the user 20 attempts to fall asleep (for example when the user 20 lays in the bed 40 reading a book); (ii) when the user 20 begins trying to fall asleep but is still awake; (iii) when the user 20 is in a light sleep (also referred to as stage 1 and stage 2 of non-rapid eye movement (NREM) sleep); (iv) when the user 20 is in a deep sleep (also referred to as slow-wave sleep, SWS, or stage 3 of NREM sleep); (v) when the user 20 is in rapid eye movement (REM) sleep; (vi) when the user 20 is periodically awake between light sleep, deep sleep, or REM sleep; or (vii) when the user 20 wakes up and does not fall back asleep.

The sleep session is generally defined as ending once the user 20 removes the user interface 120, turns off the respiratory therapy device 110, and gets out of bed 40. In some implementations, the sleep session can include additional periods of time, or can be limited to only some of the above-disclosed time periods. For example, the sleep session can be defined to encompass a period of time beginning when the respiratory therapy device 110 begins supplying the pressurized air to the airway or the user 20, ending when the respiratory therapy device 110 stops supplying the pressurized air to the airway of the user 20, and including some or all of the time points in between, when the user 20 is asleep or awake.

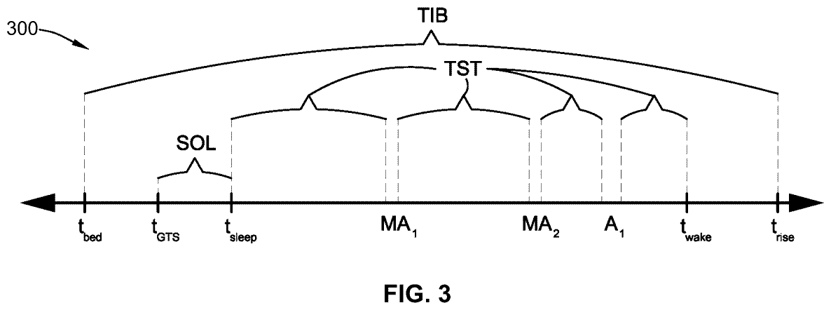

Referring to the timeline 300 in FIG. 3 the enter bed time t_{bed} is associated with the time that the user initially enters the bed (e.g., bed 40 in FIG. 2) prior to falling asleep (e.g., when the user lies down or sits in the bed). The enter bed time t_{bed} can be identified based on a bed threshold duration to distinguish between times when the user enters the bed for sleep and when the user enters the bed for other reasons (e.g., to watch TV). For example, the bed threshold duration can be at least about 10 minutes, at least about 20 minutes, at least about 30 minutes, at least about 45 minutes, at least about 1 hour, at least about 2 hours, etc. While the enter bed time t_{bed} is described herein in reference to a bed, more generally, the enter time t_{bed} can refer to the time the user initially enters any location for sleeping (e.g., a couch, a chair, a sleeping bag, etc.).

The go-to-sleep time (GTS) is associated with the time that the user initially attempts to fall asleep after entering the bed (t_{bed}). For example, after entering the bed, the user may engage in one or more activities to wind down prior to trying to sleep (e.g., reading, watching TV, listening to music, using the user device 260, etc.). The initial sleep time (tₛₗₑₑₚ) is the time that the user initially falls asleep. For example, the initial sleep time (tₛₗₑₑₚ) can be the time that the user initially enters the first non-REM sleep stage.

The wake-up time t_{wake} is the time associated with the time when the user wakes up without going back to sleep (e.g., as opposed to the user waking up in the middle of the night and going back to sleep). The user may experience one of more unconscious microawakenings (e.g., microawakenings MA₁ and MA₂) having a short duration (e.g., 5 seconds, 10 seconds, 30 seconds, 1 minute, etc.) after initially falling asleep. In contrast to the wake-up time t_{wake}, the user goes back to sleep after each of the microawakenings MA₁ and MA₂. Similarly, the user may have one or more conscious awakenings (e.g., awakening A) after initially falling asleep (e.g., getting up to go to the bathroom, attending to children or pets, sleep walking, etc.). However, the user goes back to sleep after the awakening A. Thus, the wake-up time t_{wake} can be defined, for example, based on a wake threshold duration (e.g., the user is awake for at least 15 minutes, at least 20 minutes, at least 30 minutes, at least 1 hour, etc.).

Similarly, the rising time tᵣᵢₛₑ is associated with the time when the user exits the bed and stays out of the bed with the intent to end the sleep session (e.g., as opposed to the user getting up during the night to go to the bathroom, to attend to children or pets, sleep walking, etc.). In other words, the rising time tᵣᵢₛₑ is the time when the user last leaves the bed without returning to the bed until a next sleep session (e.g., the following evening). Thus, the rising time tᵣᵢₛₑ can be defined, for example, based on a rise threshold duration (e.g., the user has left the bed for at least 15 minutes, at least 20 minutes, at least 30 minutes, at least 1 hour, etc.). The enter bed time t_{bed} time for a second, subsequent sleep session can also be defined based on a rise threshold duration (e.g., the user has left the bed for at least 4 hours, at least 6 hours, at least 8 hours, at least 12 hours, etc.).

As described above, the user may wake up and get out of bed one more times during the night between the initial t_{bed} and the final tᵣᵢₛₑ. In some implementations, the final wake-up time t_{wake} and/or the final rising time tᵣᵢₛₑ that are identified or determined based on a predetermined threshold duration of time subsequent to an event (e.g., falling asleep or leaving the bed). Such a threshold duration can be customized for the user. For a standard user which goes to bed in the evening, then wakes up and goes out of bed in the morning any period (between the user waking up (t_{wake}) or raising up (tᵣᵢₛₑ), and the user either going to bed (t_{bed}), going to sleep (t_{GTS}) or falling asleep (tₛₗₑₑₚ) of between about 12 and about 18 hours can be used. For users that spend longer periods of time in bed, shorter threshold periods may be used (e.g., between about 8 hours and about 14 hours). The threshold period may be initially selected and/or later adjusted based on the system monitoring the user's sleep behavior.

The total time in bed (TIB) is the duration of time between the time enter bed time t_{bed} and the rising time tᵣᵢₛₑ. The total sleep time (TST) is associated with the duration between the initial sleep time and the wake-up time, excluding any conscious or unconscious awakenings and/or micro-awakenings therebetween. Generally, the total sleep time (TST) will be shorter than the total time in bed (TIB) (e.g., one minute short, ten minutes shorter, one hour shorter, etc.). For example, referring to the timeline 300 of FIG. 3, the total sleep time (TST) spans between the initial sleep time tₛₗₑₑₚ and the wake-up time t_{wake}, but excludes the duration of the first micro-awakening MA₁, the second micro-awakening MA₂, and the awakening A. As shown, in this example, the total sleep time (TST) is shorter than the total time in bed (TIB).

In some implementations, the total sleep time (TST) can be defined as a persistent total sleep time (PTST). In such implementations, the persistent total sleep time excludes a predetermined initial portion or period of the first non-REM stage (e.g., light sleep stage). For example, the predetermined initial portion can be between about 30 seconds and about 20 minutes, between about 1 minute and about 10 minutes, between about 3 minutes and about 5 minutes, etc. The persistent total sleep time is a measure of sustained sleep, and smooths the sleep-wake hypnogram. For example, when the user is initially falling asleep, the user may be in the first non-REM stage for a very short time (e.g., about 30 seconds), then back into the wakefulness stage for a short period (e.g., one minute), and then goes back to the first non-REM stage. In this example, the persistent total sleep time excludes the first instance (e.g., about 30 seconds) of the first non-REM stage.

In some implementations, the sleep session is defined as starting at the enter bed time (t_{bed}) and ending at the rising time (tᵣᵢₛₑ), i.e., the sleep session is defined as the total time in bed (TIB). In some implementations, a sleep session is defined as starting at the initial sleep time (tₛₗₑₑₚ) and ending at the wake-up time (t_{wake}). In some implementations, the sleep session is defined as the total sleep time (TST). In some implementations, a sleep session is defined as starting at the go-to-sleep time (t_{GTS}) and ending at the wake-up time (t_{wake}). In some implementations, a sleep session is defined as starting at the go-to-sleep time (t_{GTS}) and ending at the rising time (tᵣᵢₛₑ). In some implementations, a sleep session is defined as starting at the enter bed time (t_{bed}) and ending at the wake-up time (t_{wake}). In some implementations, a sleep session is defined as starting at the initial sleep time (tₛₗₑₑₚ) and ending at the rising time (tᵣᵢₛₑ).

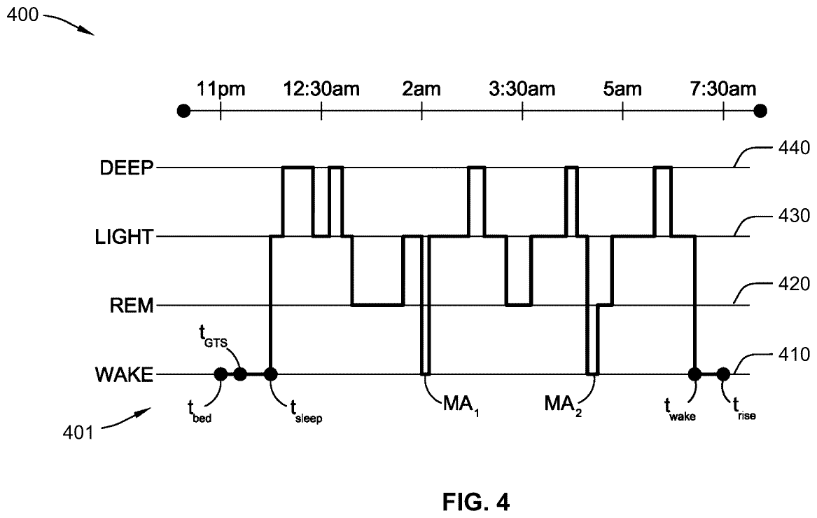

Referring to FIG. 4, an exemplary hypnogram 400 corresponding to the timeline 300 (FIG. 3), according to some implementations, is illustrated. As shown, the hypnogram 400 includes a sleep-wake signal 401, a wakefulness stage axis 410, a REM stage axis 420, a light sleep stage axis 430, and a deep sleep stage axis 440. The intersection between the sleep-wake signal 401 and one of the axes 410-440 is indicative of the sleep stage at any given time during the sleep session.

The sleep-wake signal 401 can be generated based on physiological data associated with the user (e.g., generated by one or more of the sensors 210 described herein). The sleep-wake signal can be indicative of one or more sleep states, including wakefulness, relaxed wakefulness, microawakenings, a REM stage, a first non-REM stage, a second non-REM stage, a third non-REM stage, or any combination thereof. In some implementations, one or more of the first non-REM stage, the second non-REM stage, and the third non-REM stage can be grouped together and categorized as a light sleep stage or a deep sleep stage. For example, the light sleep stage can include the first non-REM stage and the deep sleep stage can include the second non-REM stage and the third non-REM stage. While the hypnogram 400 is shown in FIG. 4 as including the light sleep stage axis 430 and the deep sleep stage axis 440, in some implementations, the hypnogram 400 can include an axis for each of the first non-REM stage, the second non-REM stage, and the third non-REM stage. In other implementations, the sleep-wake signal can also be indicative of a respiration signal, a respiration rate, an inspiration amplitude, an expiration amplitude, an inspiration-expiration ratio, a number of events per hour, a pattern of events, or any combination thereof. Information describing the sleep-wake signal can be stored in the memory device 204.

The hypnogram 400 can be used to determine one or more sleep-related parameters, such as, for example, a sleep onset latency (SOL), wake-after-sleep onset (WASO), a sleep efficiency (SE), a sleep fragmentation index, sleep blocks, or any combination thereof.

The sleep onset latency (SOL) is defined as the time between the go-to-sleep time (t_{GTS}) and the initial sleep time (tₛₗₑₑₚ). In other words, the sleep onset latency is indicative of the time that it took the user to actually fall asleep after initially attempting to fall asleep. In some implementations, the sleep onset latency is defined as a persistent sleep onset latency (PSOL). The persistent sleep onset latency differs from the sleep onset latency in that the persistent sleep onset latency is defined as the duration time between the go-to-sleep time and a predetermined amount of sustained sleep. In some implementations, the predetermined amount of sustained sleep can include, for example, at least 10 minutes of sleep within the second non-REM stage, the third non-REM stage, and/or the REM stage with no more than 2 minutes of wakefulness, the first non-REM stage, and/or movement therebetween. In other words, the persistent sleep onset latency requires up to, for example, 8 minutes of sustained sleep within the second non-REM stage, the third non-REM stage, and/or the REM stage. In other implementations, the predetermined amount of sustained sleep can include at least 10 minutes of sleep within the first non-REM stage, the second non-REM stage, the third non-REM stage, and/or the REM stage subsequent to the initial sleep time. In such implementations, the predetermined amount of sustained sleep can exclude any micro-awakenings (e.g., a ten second micro-awakening does not restart the 10-minute period).

The wake-after-sleep onset (WASO) is associated with the total duration of time that the user is awake between the initial sleep time and the wake-up time. Thus, the wake-after-sleep onset includes short and micro-awakenings during the sleep session (e.g., the micro-awakenings MA₁ and MA₂ shown in FIG. 3), whether conscious or unconscious. In some implementations, the wake-after-sleep onset (WASO) is defined as a persistent wake-after-sleep onset (PWASO) that only includes the total durations of awakenings having a predetermined length (e.g., greater than 10 seconds, greater than 30 seconds, greater than 60 seconds, greater than about 5 minutes, greater than about 10 minutes, etc.) The sleep efficiency (SE) is determined as a ratio of the total time in bed (TIB) and the total sleep time (TST). For example, if the total time in bed is 8 hours and the total sleep time is 7.5 hours, the sleep efficiency for that sleep session is 93.75%. The sleep efficiency is indicative of the sleep hygiene of the user. For example, if the user enters the bed and spends time engaged in other activities (e.g., watching TV) before sleep, the sleep efficiency will be reduced (e.g., the user is penalized). In some implementations, the sleep efficiency (SE) can be calculated based on the total time in bed (TIB) and the total time that the user is attempting to sleep. In such implementations, the total time that the user is attempting to sleep is defined as the duration between the go-to-sleep (GTS) time and the rising time described herein. For example, if the total sleep time is 8 hours (e.g., between 11 PM and 7 AM), the go-to-sleep time is 10:45 PM, and the rising time is 7:15 AM, in such implementations, the sleep efficiency parameter is calculated as about 94%.

The fragmentation index is determined based at least in part on the number of awakenings during the sleep session. For example, if the user had two micro-awakenings (e.g., micro-awakening MA₁ and micro-awakening MA₂ shown in FIG. 3), the fragmentation index can be expressed as 2. In some implementations, the fragmentation index is scaled between a predetermined range of integers (e.g., between 0 and 10).

The sleep blocks are associated with a transition between any stage of sleep (e.g., the first non-REM stage, the second non-REM stage, the third non-REM stage, and/or the REM) and the wakefulness stage. The sleep blocks can be calculated at a resolution of, for example, 30 seconds.

In some implementations, the systems and methods described herein can include generating or analyzing a hypnogram including a sleep-wake signal to determine or identify the enter bed time (t_{bed}), the go-to-sleep time (t_{GTS}), the initial sleep time (tₛₗₑₑₚ), one or more first micro-awakenings (e.g., MA₁ and MA₂), the wake-up time (t_{wake}), the rising time (tᵣᵢₛₑ), or any combination thereof based at least in part on the sleep-wake signal of a hypnogram.

In other implementations, one or more of the sensors 210 can be used to determine or identify the enter bed time (t_{bed}), the go-to-sleep time (t_{GTS}), the initial sleep time (tₛₗₑₑₚ), one or more first micro-awakenings (e.g., MA₁ and MA₂), the wake-up time (t_{wake}), the rising time (tᵣᵢₛₑ), or any combination thereof, which in turn define the sleep session. For example, the enter bed time t_{bed} can be determined based on, for example, data generated by the motion sensor 218, the microphone 220, the camera 232, or any combination thereof. The go-to-sleep time can be determined based on, for example, data from the motion sensor 218 (e.g., data indicative of no movement by the user), data from the camera 232 (e.g., data indicative of no movement by the user and/or that the user has turned off the lights) data from the microphone 220 (e.g., data indicative of the using turning off a TV), data from the user device 260 (e.g., data indicative of the user no longer using the user device 260), data from the pressure sensor 212 and/or the flow rate sensor 214 (e.g., data indicative of the user turning on the respiratory therapy device 110, data indicative of the user donning the user interface 120, etc.), or any combination thereof.

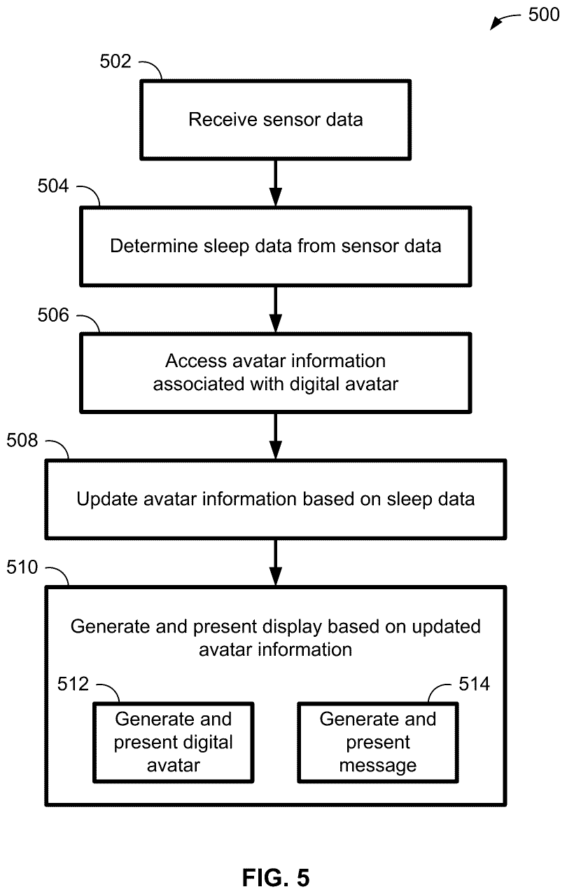

FIG. 5 is a flowchart depicting a process 500 for updating avatar information based on sleep data according to certain aspects of the present disclosure. Process 500 can be performed using system 10 of FIG. 1.

At block 502, sensor data can be received. Sensor data can be received from any suitable sensor (e.g., any of one or more sensors 210 of FIG. 1). In some cases, the sensor data received at block 502 includes i) sensor data acquired from one or more sensors of a respiratory therapy system (e.g., respiratory therapy system 100 of FIG. 1); ii) sensor data acquired from a user device (e.g., user device 260 of FIG. 1); iii) sensor data acquired from an activity tracker (e.g., activity tracker 270 of FIG. 1); iv) sensor data acquired from a blood pressure device (e.g., blood pressure device 280 of FIG. 1); or v) any combination of i-iv. The sensor data can be collected before, during, and/or after a user's sleep session. In some cases, the sensor data is received in realtime or approximate realtime, although that need not always be the case.

At block 504, sleep data can be determined from the sensor data. Sleep data can include sleep-related parameters, as disclosed in further detail herein. In some examples, sleep data includes i) sleep state information (e.g., indication of the user's sleep state over time, total time spent in any given sleep state, etc.); ii) sleep stage information (e.g., indication of the user's sleep stages over time, total time spent in any given sleep stage, etc.); iii) sleep enhancement or respiratory therapy device usage information (e.g., total time spent using the sleep enhancement or respiratory therapy device, the number of times sleep enhancement or respiratory therapy was stopped and restarted, the number of times the user interface was removed and/or replaced, etc.); iv) apnea event information (e.g., a total number of detected apnea events, an average number of apnea events per hour, indication of apnea events over time, etc.); v) device interaction information (e.g., time spent using a user device before falling asleep or attempting sleep, times when a user device was picked up and/or put down during or adjacent a sleep session, etc.); vi) any combination of i-v. Other types of sleep data can be used. In some cases, determining sleep data at block 504 includes determining additional data, such as activity data (e.g., time spent exercising during the day, number of steps taken, etc.). The sleep enhancement or therapy device may include any suitable type, such as sleep enhancement wearables (e.g., in the form of a headband, earbuds, etc.). The respiratory therapy device can be of any suitable type, such as positive airway pressure (PAP) device or non-PAP alternative treatment device (e.g., mandibular advancement appliance, positional therapy device, oral muscle training tool, etc.).

Sleep data can be information used to describe or define a user's sleep session, including the user's condition when entering and/or exiting the sleep session. For example, sleep data for a first user may indicate that the user exercised for an hour during the day prior to a sleep session, stopped using their smartphone an hour prior to starting the sleep session, used their prescribed respiratory therapy device throughout the sleep session, experienced few or no apnea events during the sleep session, and slept for a total of 8 hours. In another example, sleep data for a second user may indicate that the user did not exercise during the day prior to the sleep session, used their smartphone up until starting the sleep session and a few times during the sleep session during quick awakenings, experienced numerous apnea events, and slept for a total of 4:30 hours. The sleep data can be indicative of the first user achieving a higher quality of sleep than the second user.

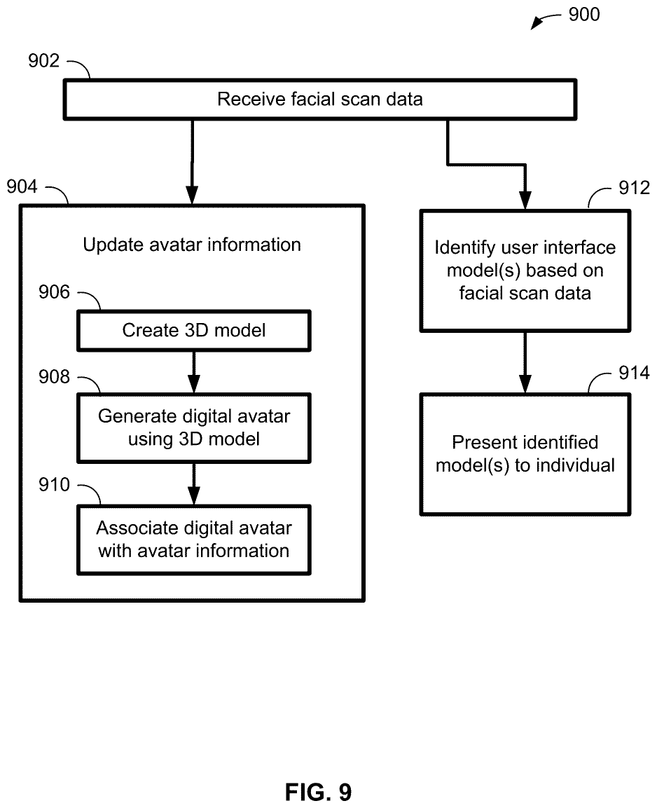

At block 506, avatar information associated with a digital avatar can be accessed. The digital avatar can be a virtual representation of the user or a character associated with the user. Such a digital avatar can be previously established during a registration process. In some cases, establishing a digital avatar can include process 900 of FIG. 9. The digital avatar can be designed to look like the user, although that need not always be the case. In some cases, different skins can be applied to a digital avatar to change its look, such as to cause a digital avatar to look like a celebrity. In some cases, the digital avatar is a virtual representation of the user. In other cases, the digital avatar can be a virtual coach that is associated with the user. The digital avatar can help urge the user to achieve certain milestones or goals, such as improved respiratory therapy compliance or improved sleep hygiene.

Generally, a single digital avatar would be associated with a single user, although that need not always be the case. In some cases, a single digital avatar may be associated with a group of individuals (e.g., a family, a support group, a team, etc.) such that sleep data from each of the different members of the group affects the digital avatar.

Avatar information associated with the digital avatar can include information used to generate a visualization (e.g., a 2-D or 3-D visualization) of the digital avatar, information used to track digital property (e.g., virtual sleepwear) associated with the avatar, information used to track achievement tokens, information used to track milestones (e.g., goals), information used to track points and/or scores (e.g., interaction points or respiratory therapy usage scores), and the like. In some cases, information used to generate a visualization can include a model that is usable to generate the digital avatar. In some cases, one or more model parameters can be applied to the model to generate the digital avatar. In some cases, the model can be updated by the one or more model parameters and the updated model can be used to generate the digital avatar. The one or more model parameters can be adjustable directly or based on other avatar information. In some cases, the one or more model parameters can be directly based on the sleep data.

In some cases, accessing the avatar information can include accessing a local storage or a remote storage (e.g., a network storage or a cloud storage).

At block 508, the avatar information can be updated based at least in part on the sleep data. Updating the avatar information can include applying the sleep data, alone or in conjunction with other data, to add, modify, or remove one or more pieces of avatar information.

In an example, sleep data indicative of high-quality sleep may be used to adjust a model parameter such that when the digital avatar is presented, it is presented with a smile and/or other features indicative of a happy or positive mood. Likewise, if the sleep data is indicative of low-quality sleep, that sleep data may be used to adjust a model parameter such that when the digital avatar is presented, it is presented with droopy eyelids and/or other features indicative of a sleepy avatar.

In some cases, updating the avatar information based on the sleep data can be based on a trained machine learning algorithm. Such an algorithm can be trained using training data from other users, and optionally historical data from the user from whom the sleep data is determined at block 504, to maximize a given goal. The goal can be improved compliance with respiratory therapy, improved sleep quality (e.g., sleep quality based on a subjective metric, such as from user feedback; or based on objective metrics, such as time in sleep or time in certain sleep stages, etc.).

At block 510, a display can be generated and presented based at least in part on the updated avatar information. The display that is generated and presented is visual content (e.g., content on a graphical user interface) that is generated and then presented to the user and/or other individuals (e.g., other users). In some cases, presenting the display can include presenting the display locally (e.g., on a display device of the user's smartphone) and/or transmitting the display for presentation on a different device (e.g., on a display device of another user's device). A display that is generated and presented can be generated by a control system (e.g., control system 200 of FIG. 1) and displayed on a display device (e.g., display device 262 or display device 150 of FIG. 1). As used herein, generating and presenting a display (e.g., a visualization of a digital avatar, a message, a profile tag associated with the digital avatar, etc.) is inclusive of updating a display that is already generated and/or already being presented. For example, reference to generating and presenting a digital avatar having a happy and alert mood is intended to include instances where the digital avatar that is already being presented with a sad and tired mood is updated to present with a happy and alert mood.

In some cases, generating and presenting the display is based on current and/or recent conditions. For example, if the updated avatar information indicates that the user experienced poor sleep the night before, the display that is generated and presented may be a visualization of the user's digital avatar taking on a tired appearance, indicating that the previous sleep session was poor. In some cases, the display can be based on an immediately previous sleep session, although that need not always be the case. In some cases, the display can be based on a number of previous sleep sessions. For example, a user that has had seven nights straight of high-quality sleep followed by a night of poor-quality sleep may have a digital avatar that takes on a less tired appearance than if that user had eight nights straight of poor-quality sleep.

In some cases, generating and presenting the display is based on predicted future conditions. For example, if the system determines that the user is achieving better quality sleep over a given time period (e.g., over the past couple weeks), the system may predict that the user will achieve even better quality sleep over the course of the next week or two and generate and present a display based on the future, better quality sleep. In some cases, the predicted future conditions can be based on user-provided or system-suggested parameters (e.g., going to sleep 30 minutes earlier per night, using the respiratory therapy device more during the night, stopping use of electronics 1 hour before starting a sleep session, etc.) rather than trends from past sleep sessions. In some cases, the system can switch between a current display (e.g., a digital avatar or message based on current and/or recent conditions) and a future display (e.g., a digital avatar or message based on predicted future conditions), or can present both together. In such cases, the system can enable a user to see how certain changes (e.g., going to sleep earlier each night) may affect the user, such as by generating and presenting a display comparing current conditions and predicted future conditions taking into account the change. In such an example, generated and presented displays may include a current digital avatar based on current conditions and a future digital avatar based on predicted future conditions. In another such example, generated and presented displays may include a message associated with current conditions (e.g., a message indicating progress towards a milestone or goal) and/or a message associated with predicted future conditions (e.g., a message indicating a milestone or goal has been achieved). In some cases, the predicted future conditions can extend out for a duration necessary to achieve a particular milestone or goal, then indicate the duration to the user. For example, a user desiring to achieve a particular milestone or goal can provide a user-provided parameter for predicting future conditions, then adjust the user-provided parameter to see how it changes and how long it will take to achieve that particular milestone or goal. For example, a user desiring to achieve fewer than four apneas per hour may adjust a parameter for how long the user makes use of respiratory therapy during the night and the system can then generate and present a display indicating when the user will achieve that goal (e.g., in one day, in 5 days, in two weeks, etc.).

In some cases, generating and presenting a display can include generating and presenting the digital avatar (e.g., a visualization of the digital avatar) at block 512. Generating the digital avatar at block 512 can include creating a visualization of the digital avatar using the updated avatar information. The visualization of the digital avatar can be a 2-dimensional or 3-dimensional visualization of some or all of the digital avatar that is based, at least in part, on avatar information. For example, avatar information can include information about digital property, such as virtual clothing and accessories that may be worn by the digital avatar, and thus included when a visualization of the digital avatar is created. In another example, avatar information can include information about achievement tokens, milestone tracking, or scores, which may affect how a visualization of the digital avatar is created (e.g., achievement tokens may be depicted on or near the digital avatar, a milestone progress score or other score may be depicted on or near the digital avatar). In some cases, the avatar information can include information about an environment or other objects associated with the digital avatar, such as a personal room, an accessory used by the digital avatar, or the like, which may affect how the visualization of these other objects are generated with respect to the digital avatar. In another example, avatar information can include information about the digital avatar's parameters or features, such as skin color, age, height, weight, mood, head size, arm size, eye color, hair color, hair style, hair length, nose length, etc., and thus can affect how the avatar is visualized.

In use, a digital avatar can be presented to the user (e.g., the individual from whom the sleep data is acquired) and/or other users. The digital avatar can be presented by itself or along with other digital avatars (e.g., digital avatars of other users). In such cases, these digital avatars can interact with one another in a virtual world. In some cases, actions taken by the digital avatars are automated (e.g., digital avatars may interact automatically and/or based on the updated avatar information from block 508) or controlled (e.g., directly controlled by a user, such as using a controller or other input device). In some cases, digital avatars may only appear with the associated user is “logged in” to the virtual world, although that need not always be the case. In some cases, a first user's digital avatar may remain in the virtual world and may be viewable by other users even if the first user is not actively participating in the virtual world. In some cases, such a digital avatar may nevertheless be generated and presented based on the first user's sleep data. In an example, if a user is currently experiencing high-quality sleep, their associated digital avatar may be present in the virtual world and may be updated to show a more positive mood and/or to show the avatar is experiencing high-quality sleep.

Digital avatars of other users can be presented to a given user based on personal information of the user such as age, gender identity, sex, sexual orientation, location, interests, hobbies, likes, dislikes, preferences, lifestyle choices (e.g., smoker, alcohol, dietary), etc. and/or different criteria, such as real-world proximity (e.g., proximity of the given user to the other users in the real world, such as living within the same city, state, or country); virtual proximity (e.g., proximity of the digital avatars within the virtual world); community membership (e.g., members of a sleep support group may be able to see each other's digital avatars); family membership (e.g., members of a family unit may be able to see each other's digital avatars; and the like. In some cases, the system can automatically assign users that experience or have experienced similar symptoms or sleep-related issues (e.g., poor sleep hygiene habits) to a collective group, in which case members of that group would be able to see each other's digital avatars. In some cases, the system can automatically assign users that use similar sleep-related equipment (e.g., similar mattresses, similar sleep enhancement or respiratory therapy devices, similar user interfaces for their respiratory therapy systems) to a collective group, in which case members of that group would be able to see each other's digital avatars. In another example, based on user's selection, the system can assign a user to a collective group of users that use different sleep-related equipment than the user. This may be useful particularly when the user is considering switching to another sleep enhancement or respiratory therapy device, and this allows the user to interact with the other users to gather information that may assist in the switching decision. In some cases, the system can automatically assign users having the same type of sleep disorder (e.g., insomnia, restless leg syndrome, parasomnia, narcolepsy, circadian rhythm sleep-wake disorders, sleep apnea) or severity of sleep disorder to a collective group. In some cases, only users that have met certain goals (e.g., earned certain badges or achievements) can be permitted to join a particular group (e.g., a VIP room) and interact with other users that have met the same goals.

In some cases, a user actively interacting (e.g., all interactions or selected interactions) with another user via their respective digital avatars can be tracked to assign interaction points. Interaction points accrued in this fashion may be used to award an achievement to a user, to provide an incentive to a user, or otherwise. This technique of promoting interactions between users can help urge a user to comply with therapy (e.g., respiratory therapy) and/or achieve good sleep hygiene, especially when interacting with other users who are experiencing or have experienced similar issues. In some cases, a user's performance (e.g., a milestone completion goal or other performance metric) can be compared with those of other users, such as other users in a group or family, to also urge the user to comply with therapy and/or achieve good sleep hygiene. In some cases, a user's badges or achievements can be showcased, which can help urge the user and/or other users to meet certain milestones or goals. For example, a “super sleeper” badge may be assigned for achieving at least a certain number of hours of sleep per night for a threshold number of consecutive days.

In some cases, use of an avatar to interact with others in a virtual world can be especially beneficial to individuals who may be shy or otherwise discouraged from interacting with others in the real world, as virtual interaction through avatars may be more comfortable and easier in certain circumstances. Further, use of an avatar to interact with others in a virtual world can be especially beneficial to individuals who are concerned with privacy, as the user can interact with others via the avatar without disclosing information about the user that the user wishes to remain private. For example, a public figure may wish to interact with others without details of their sleep, sleep-related disorder, and/or treatment being linked to themselves. In such cases, the public figure may make use of an avatar that is not outwardly associated with the public figure to interact with others.

While generally users participate in the virtual world, other entities can participate as well. In some cases, non-user participants (e.g., participants form whom sleep data is not acquired to update avatar information) can be associated with digital avatars and other digital properties. For example, a sleep accessory retailer may purchase billboard space on a digital billboard within the virtual world.

In some cases, when determining sleep data at block 504 further includes determining additional data, such as activity data, such additional data can be used to update the avatar information at block 508, and the display can be generated and presented at block 510 based on the updated avatar information that is based at least in part on the additional data. In an example, activity data, such as data indicating a user engaged in a 30-minute running exercise, can be used to update the avatar information such that the user's digital avatar is depicted as running and/or depicted as having recently exercised (e.g., showing sweat, wearing exercise clothing or accessories, appearing stronger or more muscular, etc.). As discussed above, in some cases generating and presenting the display is based on predicted future conditions. In an example where activity data is used to update avatar information, generating and presenting a display based on predicted future conditions can include presenting a digital avatar that is indicative of a health change based on activity data (e.g., historical, current, and/or predicted future activity data). In such an example, a user may see their current digital avatar and select to see what their digital avatar would look like if they engaged in an additional 30 minutes of exercise each day or if they continued exercising the same amount they have during the past several days, in which case the predicted future avatar may appear healthier, slimmer, more muscular, and/or happier. In another example, additional data can include data from a user device (e.g., user device 260 of FIG. 1) such as smartphone, such as screen time data throughout the day. When screen time is lower, the digital avatar might appear happier. However, in some cases, screen time data can be sleep data when the screen time data is associated with a sleep session (e.g., screen time data within a certain threshold amount of time before a sleep session).

Overall, the appearance of the digital avatar, and any associated objects or accessories, can be designed to motivate and/or coach a user to improve their sleep or achieve other sleep-related goals.

In some cases, generating and presenting a display can include generating and presenting a message associated with the updated avatar information at block 514. The message can be presented along with a visualization of a digital avatar (e.g., from block 512) or on its own. The message can be presented on the user's display device and/or other users' display devices.

In some cases, a message can be a comment or other information designed to urge the user to meet a certain milestone or goal, or to generally improve the user's therapy compliance and/or sleep hygiene. In some cases, the message can merely identify certain sleep data (e.g., “Your AHI is currently 8”), although that need not always be the case. In some cases, the message can be based on the sleep data, but can be generated and presented in a more conversational fashion.

In some cases, the message can be from the point of view of the digital avatar. For example, in response to a user having low quality sleep during a sleep session, a subsequent message that is generated and presented can say “I didn't have great sleep last night, so I'm going to be going to bed a bit earlier tonight.” In some cases, the message can also prompt the user to take action, such as by saying “Would you like to try going to bed earlier tonight, as well?” In some cases, the message can prompt the user to establish an alarm or further message, such as by saying “Would you like me to remind you at 8:45 pm to go to bed a bit earlier tonight?” in which case a positive response can cause the system to establish a reminder for 8:45 pm that night. In some cases, certain actions, such as suggesting a time for a reminder, can be based on sleep data. In this example, the sleep data may indicate that the previous night's sleep session, which was of low quality, commenced at 11:30 pm, so the system may urge the user to go to sleep at 9 pm-10 pm by offering to present a reminder at 8:45 pm.

In some cases, messages are indications of achievement and/or progress of milestones or goals. For example, a message can be presented when the user has achieved a desired goal (e.g., slept at least 8 hours for 30 consecutive days) or when the user is progressing towards a certain milestone (e.g., has steadily increased the nightly time spent using respiratory therapy over the course of the week).

In some cases, the display generated and presented at block 510 can be triggered to be presented at a certain time instead of on demand. In some cases, the user can select a time when the user should be notified. In some cases, a time to notify the user with the display can be automatically determined based on the user's behaviors. For example, it may be optimal to have the digital avatar appear to the user to provide coaching to help improve the next sleep session at a time close to the next sleep session (e.g., an hour before the user is expected to initiate their next sleep session, as determined from historical sleep data).

In some cases, generating and presenting a display can include generating the digital avatar along with a profile tag associated with the digital avatar. The profile tag may include information such as years on therapy, sleep score, average nightly usage, location, settings, details of sleep-related equipment being used (e.g., brand and/or type of a sleep enhancement or respiratory therapy device or type of user interface, etc.). The profile tag of the first user's digital avatar, for example, may be viewable by other users by clicking on the first user's digital avatar and vice versa. The user may customize the type and amount of information that appears in the profile tag. The information displayed in the profile tag may be manually updated by the user or dynamically updated upon detection of a change in the sensor data. The profile tag associated with the digital avatar may be used as a tool to allow the user to promote himself to other users in the virtual world, or act as a motivator for other users who are not as experienced or compliant as the user.

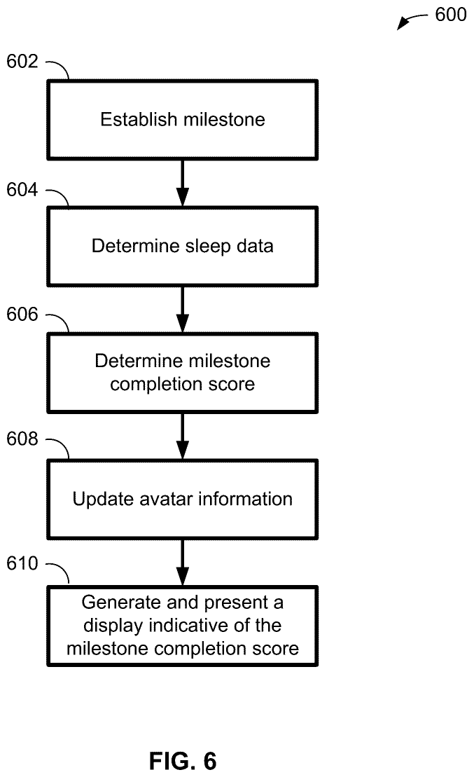

Process 500 is described herein with certain blocks in a certain order. However, in some cases, process 500 may include additional or fewer blocks, as well as blocks in different orders and/or different blocks merged or split. For example, in some cases, process 500 may include certain blocks from process 600, 700, 800, or 900 of FIGS. 6-9.

FIG. 6 is a flowchart depicting a process 600 for presenting displays related to milestone achievements based on sleep data according to certain aspects of the present disclosure. Process 600 can be performed using system 10 of FIG. 1.

At block 602, a milestone or goal can be established. Establishing a milestone or goal can be performed automatically or manually. Manual establishment of a milestone or goal can include permitting a user or practitioner (e.g., a user's caretaker, physician, nurse, or other care provider) to manually set a desired goal, such as using respiratory therapy for at least 6 hours each night. Automatic establishment of a milestone or goal can include automatically setting a milestone or goal based on historical data, such as historical sleep data (e.g., sleep data from one or more past sleep sessions). For example, if historical sleep data shows that the user regularly starts a sleep session at 11 pm-12 am, the automatic milestone or goal may be set to starting sleep sessions at 9 pm-10 pm. In another example, if historical sleep data shows that the user has poor-quality sleep during sleep sessions prefaced by more than 15 minutes of screen time within the hour before starting the sleep session, but has high-quality sleep during sleep sessions prefaced by at least an hour free from screen time before starting the sleep session, the system may automatically identify a milestone or goal of eliminating screen time during the hour prior to a sleep session.

In some cases, a user can establish an overall milestone or goal at block 602, and the system can automatically generate one or more sub-milestones or sub-goals at block 602 that can each be monitored through their own process 600.

At block 604, sleep data can be determined. Determining sleep data at block 604 can be the same as or similar to determining sleep data at block 504 of FIG. 5.

At block 606, a milestone or goal completion score can be determined. A milestone or goal completion score can be an indication of the progress of a milestone or goal. In some cases, a milestone or goal completion score is binary (e.g., 0=the milestone is not complete, 1=the milestone is complete). In some cases, a milestone or goal completion score is a percentage out of 100%. Other techniques for scoring milestone or goal progress can be used.

At block 608, avatar information can be updated based at least in part on the milestone or goal completion score. Avatar information can be updated at block 608 similar to updating avatar information at block 508 of FIG. 5, but using the milestone or goal completion score. For example, upon completing a given milestone, the avatar information may be updated to indicate that an achievement or trophy has been earned.

At block 610, a display indicative of the milestone or goal completion score can be generated and presented. Generating and presenting the display at block 610 can be similar to generating and presenting the display at block 510 of FIG. 5, but specifically based at least in part on the milestone or goal completion score. For example, if the user has achieved a certain milestone, a badge can be awarded, which can cause a display to be generated and presented that depicts the user's digital avatar wearing or adjacent to the badge, and/or that depicts a message indicating the badge has been awarded. In another example, if the milestone completion score determined at block 606 is indicative of 50% progress towards a particular milestone or goal, the display generated and presented at block 610 may be a message praising the user's progress towards the milestone or goal.

In some cases, upon determining that a milestone or goal has been completed, or that an amount of progress has been reached, the system can enable new features associated with the system. For example, after spending a threshold amount of time on therapy, the system may unlock additional features for the respiratory therapy device (e.g., additional modes of use, additional settings, etc.).

In some cases, upon determining that a milestone or goal has been completed, or that an amount of progress has been reached, the system can unlock information (e.g., articles and the like) or other media.

Process 600 is described herein with certain blocks in a certain order. However, in some cases, process 600 may include additional or fewer blocks, as well as blocks in different orders and/or different blocks merged or split. For example, in some cases, process 600 may include certain blocks from process 500, 700, 800, or 900 of FIGS. 5 and 7-9.

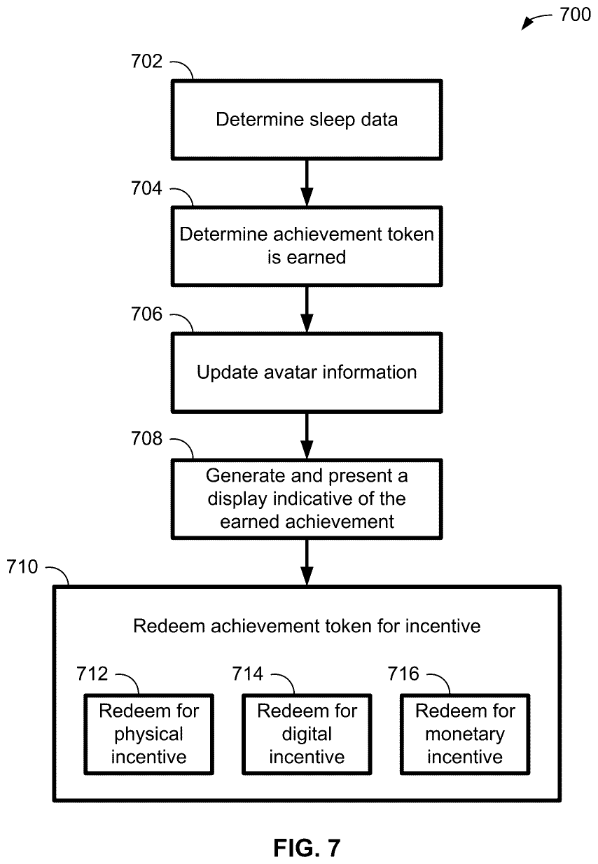

FIG. 7 is a flowchart depicting a process for managing earned achievements based on sleep data according to certain aspects of the present disclosure. Process 700 can be performed using system 10 of FIG. 1.

At block 702, sleep data can be determined. Determining sleep data at block 702 can be the same as or similar to determining sleep data at block 504 of FIG. 5.

At block 704, a determination is made that an achievement token is earned. Earning an achievement token can be based on sleep data from block 702. In some cases, determining that an achievement token is earned at block 704 can include performing some or all of the blocks of process 600. For example, when sleep data indicates that that a particular milestone has been completed, the system may determine that an achievement token is earned. The achievement token can be any suitable digital token indicating that a particular milestone or goal has been achieved or completed.

In some cases, an achievement token can be a point in a point-earning system. For example, determining that an achievement token is earned at block 704 can include determining that a point is earned and/or updating a total number of points. In some cases, points can be earned for various categories. In some cases, points may be earned for effort engaged in by the user to achieve improved sleep or otherwise achieve a milestone or goal. In an example, a user may receive points for continued use of their respiratory therapy device (e.g., over the course of a night and/or over the course of multiple sleep sessions), a total number of hours on therapy, a number of sleep-related articles read, a percentage of time the user puts their mask back on after taking it off during a sleep session, and the like.

At block 706, avatar information can be updated based at least in part on the determination that the achievement token is earned. Avatar information can be updated at block 706 similar to updating avatar information at block 508 of FIG. 5, but based on the earned achievement token. For example, upon completing a milestone related to compliant use of respiratory therapy, the avatar information may be updated to indicate that an achievement token (e.g., “PAP Pro” or “Super PAP Sleeper”) has been earned and is now associated with the user's digital avatar.

At block 708, a display can be generated and presented that is indicative of the earned achievement. Generating and presenting the display at block 708 can be similar to generating and presenting the display at block 510 of FIG. 5, but specifically based at least in part on the earned achievement token. For example, when a “PAP Pro” achievement is earned, the user's digital avatar can be generated and presented wearing or adjacent to a “PAP Pro” badge, and/or a message can be generated indicating that the “PAP” Pro” badge has been earned. In another example, when another user attempts to view information about the first user's digital avatar, the other user may be presented with a visualization of the first user's digital avatar and an indication that the “PAP Pro” badge has been earned and is associated with the first user's digital avatar (e.g., displaying the badge adjacent to or grouped with the first user's digital avatar).

In some optional cases, at block 710, an achievement token can be redeemed for an incentive. Redeeming the achievement token at block 710 can optionally burn the achievement token (e.g., the user loses the achievement token when it is redeemed), although that need not always be the case (e.g., the user retains the achievement token when it is redeemed, although it may be restricted from being redeemed again).

In some cases, redeeming the achievement token at block 710 includes redeeming the achievement token for a physical incentive at block 712. Redeeming for a physical incentive includes receiving a physical incentive in the real world. For example, a user may redeem an achievement token for a product, such as sleep accessory or a new user interface for their respiratory therapy device.

In some cases, redeeming the achievement token at block 710 includes redeeming the achievement token for a digital incentive. A digital incentive is any computer-deliverable incentive that is usable in the virtual world or in association with the digital avatar. For example, a digital incentive may be a piece of virtual clothing for the digital avatar, an accessory to be worn by the digital avatar, a new skin for the digital avatar, new features for the digital avatar, and the like. In some cases, a digital incentive can include a non-fungible token that is created for and/or transferred to the user. In some cases, a digital incentive can be an achievement token earned by another user. In such cases, redeeming an achievement token can be a form of trading for a different achievement token.

In some cases, redeeming the achievement token at block 710 includes redeeming the achievement token for a monetary incentive at block 716. A monetary incentive can be a physical or digital product that is usable in the real world and carries a monetary value. Examples of monetary incentives include checks, digital money transfers (e.g., payment in digital currency, such as cryptocurrency, or money wire to a bank account), digital or physical coupons or coupon codes (e.g., for a discount at a retailer), donations (e.g., donations to a third-party charity).

Generally, redeeming an achievement token for an incentive at block 710 is manually initiated by the user, although that need not always be the case. In some cases, the user can set the system up to automatically redeem certain earned achievements for certain incentives. For example, a user can set up the system to automatically redeem all achievement tokens or all sleep-duration-related achievement tokens for donations to a charity, in which case earning such an achievement will automatically result in a donation being made to the charity. In some cases, automatic redeeming will nevertheless prompt the user for confirmation before redemption is complete.

In some cases, the use of achievement tokens (e.g., points or other tokens) can be used to identify particularly high-scoring avatars, and thus particularly high-scoring users. These high-scoring users can be provided special privileges and/or offers. For example, in some cases input from a high-scoring user may be considered more reliable. As another example, entities performing research may contact high-scoring users to offer participation in research, such as to receive and/or test new products.

Process 700 is described herein with certain blocks in a certain order. However, in some cases, process 700 may include additional or fewer blocks, as well as blocks in different orders and/or different blocks merged or split. For example, in some cases, process 700 may include certain blocks from process 500, 600, 800, or 900 of FIGS. 5-6 and 8-9. In another example, process 700 may include only one of block 708 and block 710, or may include both of block 708 and block 710 in a different order in no order.

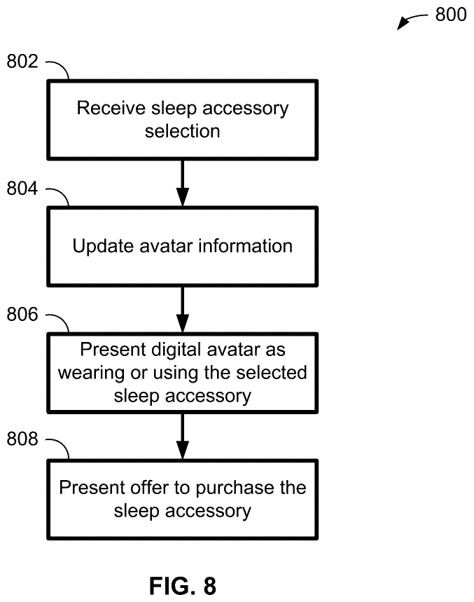

FIG. 8 is a flowchart depicting a process for presenting a digital avatar with a sleep accessory according to certain aspects of the present disclosure. Process 800 can be performed using system 10 of FIG. 1.

At block 802, a sleep accessory selection can be received. Receiving a sleep accessory selection can include receiving user input indicative of a sleep accessory to be associated with the digital avatar. The sleep accessory selection can be based on a list of sleep accessories presented to the user, such as a list of possible respiratory therapy user interfaces.

At block 804, avatar information can be updated based at least in part on the selected sleep accessory from block 802. Avatar information can be updated at block 804 similar to updating avatar information at block 508 of FIG. 5, but based on the sleep accessory selection from block 802. For example, upon selecting a particular model of user interface for a respiratory therapy system, the avatar information can be updated to associate that model of user interface with the user's digital avatar.

At block 806, a display can be generated and presented based at least in part on updated avatar information that was updated based at least in part on the selected sleep accessory. Generating and presenting the display at block 806 can be similar to generating and presenting the display at block 510 of FIG. 5, but specifically based at least in part on the selected sleep accessory. In the example above, since the user selected a particular model of user interface, the digital avatar can be generated and presented as wearing and/or using that model of user interface. For example, on a three-dimensional visualization of the digital avatar, a three-dimensional model of that particular model of user interface can be applied to the visualization of the digital avatar to make it seem as if the digital avatar was wearing the user interface.

In some optional cases, at block 808, an offer can be presented to purchase the selected sleep accessory. Upon confirming the offer to purchase the selected sleep accessory, the user can be sent a real-world product of the selected sleep accessory. In the example above, the system can offer the selected user interface for purchase, and the user can accept the offer, resulting in a real-world user interface being shipped to the user. In some cases, instead of presenting an offer to purchase the sleep accessory at block 808, process 800 can include otherwise facilitating procurement and/or purchasing of the real-world sleep accessory.

Process 800 is described herein with certain blocks in a certain order. However, in some cases, process 800 may include additional or fewer blocks, as well as blocks in different orders and/or different blocks merged or split. For example, in some cases, process 800 may include certain blocks from process 500, 600, 700, or 900 of FIGS. 5-7 and 9.

FIG. 9 is a flowchart depicting a process for using facial scan data according to certain aspects of the present disclosure. Process 900 can be performed using system 10 of FIG. 1.

At block 902, facial scan data can be received. Facial scan data can be obtained from one or more sensors, such as the one or more sensors used with respect to block 502 of FIG. 5, or other sensor(s). In some cases, the facial scan data is received from a camera, IR camera, ranging device (e.g., LIDAR), or the like. Once obtained, the facial scan data can be used for various purposes.

In some cases, the facial scan data can be used to update avatar information at block 904. Updating the avatar information at block 904 is inclusive of initial generation of avatar information. Updating the avatar information at block 904 can include using the facial scan data to either i) initially establish a digital avatar, or ii) update an existing digital avatar. When initially establishing a digital avatar, updating avatar information at block 904 can include creating a three-dimensional model of the user's face from the facial scan data at block 906; generating a digital avatar based on the three-dimensional model at block 908 (e.g., mapping the three-dimensional model of the user's face to the face of the digital avatar); and associating the digital avatar with avatar information at block 910 (e.g., associating the digital avatar with existing avatar information or initially generating avatar information and associating the digital avatar with that avatar information). When updating an existing digital avatar, the facial scan data can be used to replace or modify the existing digital avatar's existing facial features, although other uses of the facial scan data can also be made.

In some cases, at block 912, a user interface can be identified based at least in part on the facial scan data. Identifying a user interface can include identifying a user interface that a user is wearing, or identifying a user interface that the system believes would fit or best fit the user's face based on the facial scan data. In some cases, the identified user interface can be used for purposes associated with the digital avatar, such as used as a selected sleep accessory as described in further detail in process 800 of FIG. 8. In some cases, however, the identified user interface can be presented to the user at block 914, thus showing the user which user interface may be best for that user to use, or helping the user identify an existing user interface. In some cases, identifying a user interface at block 912 can include identifying one or more user interface models, and presenting at block 914 can include presenting the one or more identified models. Presenting at block 914 can include presenting the user interface model textually or visually (e.g., with a 2-dimensional or 3-dimensional visualization).

As described above, the facial scan data of the user can be used to generate the digital avatar. Thus, the digital avatar can closely mirror or can be the virtual representation of the user, making the digital avatar more personalized as well. As actual facial scan data is used, the expression of the digital avatar can closely represent the user or be more realistic, letting the user see how he or she looks like or will look like depending on the sleep data or response to the therapy.

Process 900 is described herein with certain blocks in a certain order. However, in some cases, process 900 may include additional or fewer blocks, as well as blocks in different orders and/or different blocks merged or split. For example, in some cases, process 900 may include certain blocks from process 500, 600, 700, or 800 of FIGS. 5-8.

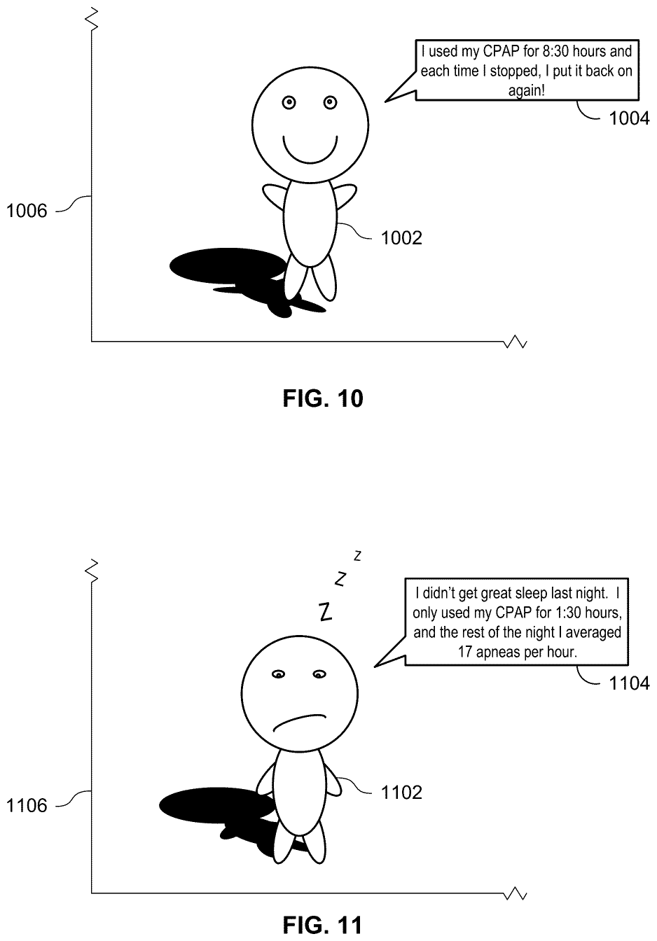

FIG. 10 is a simplified illustration of a graphical user interface (GUI) 1006 displaying a digital avatar 1002 following a high-quality sleep session, according to certain aspects of the present disclosure. The GUI 1006 can be presented on any suitable display device, such as display device 262 or display device 150 of FIG. 1. The GUI 1006 can be generated by control system 200 based on process 500, 600, 700, 800, or 900 of FIGS. 5-9.

As depicted in FIG. 10, the digital avatar 1002 is shown in a generally alert, happy, and excited mood, as generally shown by the digital avatar 1002 jumping in the air, smiling, and having open eyes. Other actions and features may be used to evoke various moods or states of the digital avatar. The digital avatar 1002 was generated based at least in part on sleep data, which sleep data indicated that the user achieved high-quality sleep or otherwise met a desired milestone or goal. In the example of FIG. 10, the GUI 1006 further presents a message 1004. Here, the message 1004 is written from the digital avatar's point of view, pointing out certain sleep data that led to the digital avatar's positive state. Specifically, the message 1004 is indicating that the digital avatar 1002 used their respiratory therapy system for 8:30 hours, including putting their user interface back on their face whenever they removed it during the sleep session.

FIG. 11 is a simplified illustration of a graphical user interface 1106 displaying a digital avatar following a low-quality sleep session, according to certain aspects of the present disclosure. The GUI 1106 can be presented on any suitable display device, such as display device 262 or display device 150 of FIG. 1. The GUI 1106 can be generated by control system 200 based on process 500, 600, 700, 800, or 900 of FIGS. 5-9. Digital avatar 1102 can be digital avatar 1002 after experiencing a low-quality sleep session.

As depicted in FIG. 11, the digital avatar 1102 is shown in a generally sad and tired mood, as generally shown by the digital avatar 1102 having shrugged shoulders, a slight frown, droopy eyelids, and the trailing Z's. Other actions and features may be used to evoke various moods or states of the digital avatar. The digital avatar 1102 was generated based at least in part on sleep data, which sleep data indicated that the user achieved low-quality sleep or otherwise failed to meet a desired milestone or goal. For example, the user may have stopped using their respiratory therapy device early during the sleep session and may have experienced many apneas, negatively impacting their quality of sleep. In the example of FIG. 11, the GUI 1106 further presents a message 1104. Here, the message 1104 is written from the digital avatar's point of view, pointing out certain sleep data that led to the digital avatar's negative state. Specifically, the message 1104 is indicating that the digital avatar 1102 only used their respiratory therapy system for 1:30 hours, after which they averaged 17 apneas per hour for the remainder of the sleep session.

While message 1104 (and indeed message 1004 of FIG. 10) may be written from the digital avatar's point of view, that need not always be the case. Further, in some cases, such messages may be written as suggestions or comments designed to urge the user towards achieving high-quality sleep and/or otherwise meeting their milestones or goals. For example, instead of talking about the number of hours spent using CPAP and number of apneas per hour, message 1104 may be written to suggest the user engage in a technique designed to urge the user to use their CPAP for longer during the sleep session.

One or more elements or aspects or steps, or any portion(s) thereof, from one or more of any of claims 1 to 28 below can be combined with one or more elements or aspects or steps, or any portion(s) thereof, from one or more of any of the other claims 1 to 28 or combinations thereof, to form one or more additional implementations and/or claims of the present disclosure.

While the present disclosure has been described with reference to one or more particular embodiments or implementations, those skilled in the art will recognize that many changes may be made thereto without departing from the spirit and scope of the present disclosure. Each of these implementations and obvious variations thereof is contemplated as falling within the spirit and scope of the present disclosure. It is also contemplated that additional implementations according to aspects of the present disclosure may combine any number of features from any of the implementations described herein.

1. A method comprising:
receiving sensor data from one or more sensors, the sensor data being associated with a sleep session of an individual;
determining sleep data for the sleep session based at least in part on the sensor data;
accessing avatar information associated a digital avatar that is associated with the individual;
updating the avatar information based at least in part on the sleep data; and
generating and presenting a display based at least in part on the updated avatar information.

2. (canceled)

3. The method of claim 1, wherein generating and presenting the display includes generating and presenting the digital avatar, and wherein the digital avatar based at least in part on the updated avatar information is visually distinguishable from the digital avatar prior to updating the avatar information.

4. The method of claim 1, wherein the avatar information includes an avatar model usable to generate the digital avatar, wherein updating the avatar information includes updating the avatar model usable to generate the digital avatar.

5. The method of claim 1, wherein generating and presenting the display includes:
generating a message based at least in part on the sleep data; and
presenting the message.

6. The method of claim 1, wherein determining the sleep data includes determining a respiratory therapy score associated with use of a respiratory therapy system by the individual during the sleep session, and wherein updating the avatar information is based at least in part on the respiratory therapy score.

7. The method of claim 1, further comprising establishing a milestone associated with the individual, wherein updating the avatar information includes updating a milestone completion score associated with the established milestone based at least in part on the sleep data.

8. The method of claim 7, wherein establishing the milestone is based at least in part on historical sleep data associated with a previous sleep session.

9. The method of claim 7, wherein generating and presenting the display includes:
determining that the milestone has been achieved based at least in part on updated milestone completion score; and
generating the display in response to determining that the milestone has been achieved.

10. The method of claim 9, wherein determining that the milestone has been achieved is further based at least in part on historical sleep data associated with a previous sleep session.

11. The method of claim 7, wherein generating and presenting the display includes:
determining that the milestone completion score falls below a threshold score;
generating a message in response to determining that the milestone completion score falls below the threshold score; and
presenting the message.

12. The method of claim 1, wherein updating the avatar information includes:
determining that an achievement token is earned based at least in part on the sleep data; and
updating the avatar information to indicate that the digital avatar has earned the achievement token.

13. (canceled)

14. The method of claim 1, wherein generating and presenting the display includes presenting the digital avatar to a user other than the individual

15. The method of claim 1, wherein updating the avatar information includes i) modifying a visual representation of a mood of the digital avatar; ii) modifying a virtually-wearable accessory associated with the digital avatar; iii) modifying an achievement indicator associated with the digital avatar; iv) causing the digital avatar to perform a predefined action when presented; or v) any combination of i-iv.

16. The method of claim 1, wherein the sleep data includes i) a sleep state; ii) a sleep stage; or iii) both i and ii, wherein updating the avatar information based at least in part on the sleep data includes updating the avatar information based at least in part on i) the sleep state; ii) the sleep stage; or iii) both i and ii.

17. The method of claim 1, further comprising:
receiving additional sensor data from the one or more sensors or one or more additional sensors, the additional sensor data being associated with activity of the individual outside of the sleep session;
determining activity data based at least in part on the additional sensor data; and
updating the avatar information based at least in part on the determined activity data.

18. The method of claim 1, further comprising:
receiving a selection for a sleep accessory; and
updating the avatar information based on the selection for the sleep accessory, wherein generating and presenting the display includes presenting the digital avatar as wearing the selected sleep accessory.

19. The method of claim 18, further comprising presenting an offer to purchase the sleep accessory in association with the presented digital avatar wearing the selected sleep accessory.

20. The method of claim 1, further comprising:
receiving facial scan data from the one or more sensors or from one or more additional sensors, the facial scan data being associated with a face of the individual; and
updating the avatar information based at least in part on the received facial scan data.

21. The method of claim 20, wherein updating the avatar information based at least in part on the received facial scan data includes:
creating a three-dimensional model based at least in part on the received facial scan data;
generating the digital avatar using the three-dimensional model; and
associating the generated digital avatar with the avatar information.

22. The method of claim 20 further comprising:
identifying a set of one or more user interface models intended to fit the individual based at least in part on the facial scan data; and
presenting the set of one or more user interface models to the individual.

23. The method of claim 1 further comprising:
for each of one or more participants, receiving participant avatar information associated with a respective participant avatar, wherein the respective participant avatar information is updated based at least in part on respective sleep data for the respective participant; and
presenting the one or more participant avatars in conjunction with presenting the display.

24. A system comprising:
a control system including one or more processors; and
a memory having stored thereon machine readable instructions;
wherein the control system is coupled to the memory, and the method of claim 1 is implemented when the machine executable instructions in the memory are executed by at least one of the one or more processors of the control system.

25-27. (canceled)

28. A system, comprising:
a control system comprising one or more processors;
one or more sensors coupled to the control system to provide sensor data associated with a sleep session of an individual to the one or more processors;
a display device communicatively coupled to the control system; and
a non-transitory computer readable medium having thereon machine executable instruction, which, when executed by the one or more processors, cause the control system to perform operations including:
receiving the sensor data from one or more sensors;
determining sleep data for the sleep session based at least in part on the sensor data;
accessing avatar information associated a digital avatar that is associated with the individual;
updating the avatar information based at least in part on the sleep data; and
generating and presenting a display based at least in part on the updated avatar information, wherein the display is presented on the display device.
